(19) United States
(12) Patent Application Publication
(10) Pub. No.: US 2020/0215204 A1
(43) Pub. Date: Jul. 9, 2020

(54) EARLY-ONSET PARKINSON'S DISEASE MODEL: (D331Y) PLA2G6 KNOCKIN MODEL, PLATFORM AND METHOD FOR DRUG SCREENING, AND KIT OF DETECTION

(71) Applicants: Chang Gung Memorial Hospital, Linkou, Taoyuan City (TW); Chang Gung University, Taoyuan City (TW)

(72) Inventors: Ching-Chi CHIU, Taoyuan City (TW); Tu-Hsueh YEH, Taipei City (TW); Hung-Li WANG, Taoyuan City (TW)

(21) Appl. No.: 16/527,152

(22) Filed: Jul. 31, 2019

(30) Foreign Application Priority Data
Jan. 3, 2019 (TW) 108100196

(51) Int. Cl. A61K 49/00; C12Q 1/6883; A01K 67/027

(52) U.S. Cl. CPC A61K 49/0008; C12Q 1/6883; A01K 67/0278; A01K 2267/0318; A01K 2217/072; A01K 2227/105; C12Q 2600/158

(57) ABSTRACT

Disclosed is a (D331Y) PLA2G6 knockin mouse, which shows similar clinical symptoms to those of patients suffering from Parkinson's disease (PD), and begins to display early-onset cell death of dopaminergic neurons in its substantia nigra (SN), synucleinopathy, and tau pathology at the age of about 6 months, wherein the dopaminergic neurons exhibit mitochondrial structural abnormality and dysfunction. Treatment of the (D331Y) PLA2G6 knockin mouse with L-Dopa shows a good response. The (D331Y) PLA2G6 knockin mouse can be used as a platform for developing a medicament and method for treating PD.

### RELATED DISCLOSURE

The contents of the present invention were already in part on-line published on Aug. 8, 2018 in the Journal “Molecular Neurobiology” (website: https://doi.org/10.1007/s12035-018-1118-5).

### FIELD OF THE INVENTION

The present invention relates to a homozygous (D331Y) PLA2G6 knockin mouse, a platform and method of using the homozygous (D331Y) PLA2G6 knockin mouse for screening of drugs for treating early-onset Parkinson's disease, and a kit and model of detecting the presence of homozygous (D331Y) PLA2G6 mutation for determining the development of early-onset Parkinson's disease.

### BACKGROUND OF THE INVENTION

Parkinson's disease (PARK; PD) is a common neurodegenerative disorder caused by progressive degeneration of dopaminergic neurons in substantia nigra pars compacta (SNpc). Clinical symptoms of PD include trembling, slowness of movement, rigidity, and balance impairment. The pathological hallmark of PD is Lewy bodies in surviving SNpc dopaminergic cells. Although the exact etiology of PD remains unveiled, mitochondrial dysfunction, endoplasmic reticulum (ER) stress, inflammation, oxidative stress and impaired autophagy are believed to be implicated in the pathogenesis of PD.

Until now, the exact mechanism of PD is still unclear. Current treatment of PD can improve its symptoms, but cannot delay progression of neurodegeneration.

Most of PD is sporadic. However, based on genetic research, it was found from about 5% to about 10% of patients suffering from heredofamilial PD that there were more than 10 genetic variations associated with PD. There is almost no difference in clinical symptoms and pathological changes between the patients suffering from heredofamilial PD and the patients suffering from sporadic PD. Incidence of heredofamilial PD and sporadic PD may be induced by the same molecular mechanism resulting in neurodegeneration.

Patients affected with autosomal recessive PARK14 display early-onset parkinsonism. Mutation of PLA2G6 (Ca²⁺-independent phospholipase A₂ group 6) gene causes PLA2G6-associated neurodegeneration (PLAN), including infantile neuroaxonal dystrophy, atypical neuroaxonal dystrophy, neurodegeneration with brain iron accumulation, and young-onset PARK14. Both of patients suffering from PARK14 and patients suffering from sporadic PD display the same clinical symptoms. Previous studies reported that homozygous (D331Y) mutation in PLA2G6 is the genetic cause of PARK14 patients (Shi C. H. et al., Neurology, 77(1):75-81). Patients with homozygous (D331Y) PLA2G6 mutation display motor dysfunctions of pure early-onset autosomal-recessive PD, and patients with heterozygous (D331Y) mutation in PLA2G6 have an increased risk in the development of early-onset PD (Lu C. S. et al., Am J Med Genet 2012, 159B:183-191).

PLA2G6 participates in several cellular functions, such as mitochondrial function, fatty acid oxidation, calcium signaling, cell growth, apoptosis and gene regulation. Detection of PLA2G6 gene was used for diagnosing infantile neuroaxonal dystrophy, rather than early-onset parkinsonism.

Until now, detection of mutation in PLA2G6 gene has not been used for clinical diagnosis of PD, especially PARK14.

Though genetic research has provided further understanding in pathogenic causes and mechanisms of PD, and also a basis and guideline in developing new therapies in neuroprotection against PD, however current animal models of PD include transgenic mice and knockout mice.

Transgene and gene knockout approaches have been used for investigating a relationship between gene variations and neurological diseases; however, there still exist potential problems in the transgene and gene knockout approaches:

(1) incomplete deletion of a target gene:
(A) due to insertion of a selection marker into the target gene, unaffected gene fragments can be expressed for producing (poly)peptides that are undesirable;
(B) expression of the target gene may be regulated by other promoters or starting codon (AUG) so that expression of the target gene is still possible; and
(C) abnormal exons may be produced;

(2) deletion of other gene(s):
for deleting the target gene, an expression domain comprising the target gene may wholly deleted, wherein the other gene(s) may be also deleted, so that a function of the target gene cannot be actually recognized;

(3) influence of the selected gene:
after homologous recombination, the selected gene may affect phenotype;

(4) current transgenic and knockout mice models of PD fail to display early-onset neurodegeneration or death of dopaminergic neurons in substantia nigra pars compacta (SNpc):
(A) 14-month old PLA2G6 knockout mice do not display death of dopaminergic neurons in substantia nigra pars compacta (SNpc) (Beck G et al., PLoS One. 2016, 11: e0153789);
(B) 12-month old G2019S LRRK2 knockin mice do not display death of dopaminergic neurons in substantia nigra pars compacta (SNpc) (Longo F. et al., Acta Neuropathol Commun., 2017, 5:22), and until 15-month old, LRRK2 knockout mice display death of dopaminergic neurons in substantia nigra pars compacta (SNpc) (Giaime E. et al., Neuron. 2017, 96:796-807);
(C) 8- to 12-month old PINK1 knockout mice do not display death of dopaminergic neurons in substantia nigra pars compacta (SNpc) (Kitada T. et al., PNAS. 2007, 104:11441-6; Madeo G et al., Mov Disord. 2014, 29:41-53; Akundi R. S. et al., PLoS One. 2011, 6:e16038); and
(D) 12- to 14-month old Parkin knockout mice do not display death of dopaminergic neurons in substantia nigra pars compacta (SNpc) (Pickrell A. M. et al., Neuron. 2015, 87(2):371-81; Dai Y. et al., Mitochondrion 2013, 13:282-291; Goldberg M. S. et al., J Biol Chem. 2003, 278:43628-35); and

(5) in neurotoxin MPTP-induced mice model of PD, a significant therapeutic effect could be observed after administration of 25 mg/kg L-DOPA for 21 days (Zhao T. T. et al., BMC Complement Altern Med. 2017, 17:449; Zhao T. T. et al., Neuroscience. 2016, 339:644-654).

In contrast, knockin mice model can exactly express point mutation in a target gene and also simulate a mechanism resulting in neurodegeneration induced by said point mutation without affecting expression of the other genes.

A knockin mice model harboring a point mutation in a target gene is needed in the art.

Currently, there is no animal model of PD that displays early-onset parkinsonism. Up to now, a knockin mouse expressing PARK14 (D331Y) PLA2G6 has not been developed for investigating the pathogenic mechanism of (D331Y) PLA2G6-induced PD.

### DISCLOSURE OF THE INVENTION

The present invention discloses a homozygous (D331Y) PLA2G6 knockin mouse, a platform and method of using the homozygous (D331Y) PLA2G6 knockin mouse for screening of drugs for treating early-onset Parkinson's disease, and a kit and model of detecting the presence of homozygous (D331Y) PLA2G6 mutation for determining the development of early-onset Parkinson's disease.

In an aspect, based on the PLA2G6 D331Y mutation, the present invention discloses a homozygous PLA2G6^{D331Y/D331Y} knockin mouse that can be used as an animal model for simulating a genetic variation of (D331Y) PLA2G6 in PARK14 patients, wherein the homozygous PLA2G6^{D331Y/D331Y} knockin mouse expresses a significantly low level of PLA2G6 and simultaneously displays early-onset degeneration and death of dopaminergic neurons in substantia nigra pars compacta (SNpc), synucleinopathy and tau pathology.

Under pathophysiological observation, the homozygous PLA2G6^{D331Y/D331Y} knockin mouse displays:

(1) early-onset degeneration of dopaminergic neurons in substantia nigra pars compacta (SNpc):
current animal models of PD display late-onset neurodegeneration, or neurodegeneration until about 12 months old; in contrast, the homozygous PLA2G6^{D331Y/D331Y} knockin mouse displays early-onset neurodegeneration at about 6 months old, wherein dopaminergic neurons was significantly reduced in the SNpc, and ER stress-related proteins were over-expressed;

(2) synucleinopathy and tau pathology:
current transgenic or knockout mice models of PD display Lewy bodies in surviving SNpc dopaminergic cells at about 12 months old; in contrast, the homozygous PLA2G6^{D331Y/D331Y} knockin mouse simultaneously displays synucleinopathy and tau pathology, and accumulation of Lewy bodies in surviving SNpc dopaminergic cells at about 9 months old; and, expression of a high level of phosphorylated tau protein could be found;

(3) early-onset parkinsonism:
current transgenic or knockout mice models of PD display motor dysfunction or motor deficit at about 12 months old; in contrast, the homozygous PLA2G6^{D331Y/D331Y} knockin mouse displays significant motor dysfunction or motor deficit at about 6 to 9 months old;

(4) abnormality in the ultrastructure of mitochondria, and mitochondrial dysfunction:
the homozygous PLA2G6^{D331Y/D331Y} knockin mouse displays abnormality in the structure of myelin, disrupted structure of mitochondria cristae, and change in mitochondrial morphology in dopaminergic neurons at about 9 months old; and, the homozygous PLA2G6^{D331Y/D331Y} knockin mouse displays a reduced activity of mitochondrial complex I so as not to produce an adequate amount of ATP; over-production of reactive oxygen species (ROS) renders mitochondria up-regulating lipid peroxidation and producing an increased level of cytochrome c, resulting in apoptosis of dopaminergic neurons; in addition, the homozygous PLA2G6^{D331Y/D331Y} knockin mouse displays mitochondrial mitophage dysfunction due to low expression of mitochondrial mitophage-related proteins, indicating the homozygous PLA2G6^{D331Y/D331Y} mutation affects mitochondrial mitophage function;

(5) good response to the treatment with L-Dopa:
L-Dopa has been conventionally used for treating PD in clinic; administration of a lower amount of about 1.5 mg/kg of L-Dopa to the homozygous PLA2G6^{D331Y/D331Y} knockin mouse displaying motor deficit at about 9 months old can significantly improve symptoms of said motor deficit; thus, the homozygous PLA2G6^{D331Y/D331Y} knockin mouse can be used as an animal model and platform for screening of active agents or drugs for treating early-onset Parkinson's disease, especially PARK14; and

(6) specific signaling pathways:
by microarray analysis of transcriptomes of the homozygous PLA2G6^{D331Y/D331Y} knockin mouse, the homozygous PLA2G6^{D331Y/D331Y} knockin mouse displays a change in several specific genes, including those of regulating survival of, differentiation of, and apoptosis signaling in dopaminergic neurons; in particular, the homozygous PLA2G6^{D331Y/D331Y} knockin mouse displays a significantly reduced level of Catenin beta-1 encoded by CTNNB1 gene, indicating that PLA2G6 regulates expression of said CTNNB1 gene.

The homozygous PLA2G6^{D331Y/D331Y} knockin mouse according to the present invention can used for solving the following problems:

(1) understanding of the molecular mechanism of early-onset PARK14:
death of dopaminergic neurons induces parkinsonism; early prophylaxis for preventing from neurodegeneration can maintain patients' body function and living quality; the homozygous PLA2G6^{D331Y/D331Y} knockin mouse is the first animal model of early-onset PD, which can early display neurodegeneration and parkinsonism at about 6 months, and in turn can be used for understanding the molecular mechanism of early-onset PD;

(2) development of therapeutic targets and biological labels:
the homozygous PLA2G6^{D331Y/D331Y} knockin mouse displays abnormality in the ultrastructure of mitochondria, and mitochondrial dysfunction in dopaminergic neurons; the abnormal mitochondria can be used as a therapeutic target; the homozygous PLA2G6^{D331Y/D331Y} knockin mouse can be used as an animal model for developing an active agent for protecting mitochondria in dopaminergic neurons; further, the homozygous PLA2G6^{D331Y/D331Y} knockin mouse can be used as an animal model for developing biological labels that change in a level in different phases of early-onset PD, wherein the biological labels may help early diagnosis and therapy of PD; and

(3) a platform for screening of active agents for protecting dopaminergic neurons; the homozygous PLA2G6^{D331Y/D331Y} knockin mouse simultaneously displays synucleinopathy and tau pathology, and in turn can be used as a platform for screening of active agents for protecting dopaminergic neurons and also detecting a change in a level of molecules in early-onset PD.

In another aspect, the present invention discloses a platform for screening of active agents for treating motor neuron diseases, such as Parkinson's disease, in particular PARK14, comprising the homozygous PLA2G6^{D331Y/D331Y} knockin mouse that simultaneously displays early-onset degeneration of dopaminergic neurons in substantia nigra pars compacta (SNpc), synucleinopathy and tau pathology.

The present invention also provides a method for screening of active agents for treating motor neuron diseases, such as Parkinson's disease, in particular PARK14, comprising the steps of (1) administering a candidate agent to the homozygous PLA2G6^{D331Y/D331Y} knockin mouse, and (2) evaluating effects of the candidate agent in improving symptoms of motor dysfunction or motor deficit in said homozygous PLA2G6^{D331Y/D331Y} knockin mouse. The effects in improving symptoms of motor dysfunction or motor deficit include: increased level of PLA2G6, reduced degeneration or death of dopaminergic neurons, reduced synucleinopathy and tau pathology, reduced accumulation of Lewy bodies, increased level of mitochondrial complex I, increased ATP synthesis, reduced level of ROS, increased expression level of growth- and protection-related proteins of dopaminergic neurons, reduced expression of proteins relating to apoptosis of dopaminergic neurons, and increased expression of Catenin beta-1, wherein the growth- and protection-related proteins of dopaminergic neurons include one or more of Bmp6, Ccnd2, Ctnnb1, Hspa1b, Kidins220, Mapk1, Psap and Sdc2, and the proteins relating to apoptosis of dopaminergic neurons include one or both of Mark4 and Xaf1.

In the other aspect, for early diagnosis of early-onset PD, in particular PARK14, mutation in PLA2G6 gene can be detected for determining the presence of G991T mutation.

Genomic DNA can be extracted from a blood sample of patients suspected of suffering from early-onset PD.

One of the following pairs of forward (F) primer and reverse (R) primer can be used for performing polymerase chain reaction (PCR) amplification of a coding region of PLA2G6 gene:

```
(SEQ ID NO. 1)
  1) F primer: gacagggccaccagtgattg;
(SEQ ID NO. 2)
        R primer: agttcgagatgagacacgggc;
(SEQ ID NO. 3)
  2) F primer: caggatctggggacaacgc;
(SEQ ID NO. 4)
        R primer: gccaataagacctccaatcc;
(SEQ ID NO. 5)
  3) F primer: gggaccttctgattccagc;
(SEQ ID NO. 6)
        R primer: gcccacacaagcaggtacac;
(SEQ ID NO. 7)
  4) F primer: aaagtccgagtttccgagtg;
(SEQ ID NO. 8)
        R primer: aggcctgagagtgacacctg;
(SEQ ID NO. 9)
  5) F primer: cccggcctctttacgttc;
(SEQ ID NO. 10)
        R primer: ctcaggcacgggacagg ;
(SEQ ID NO. 11)
  6) F primer: cttcatcccacgccacg;
(SEQ ID NO. 12)
        R primer: gaacctgcttcctgaggg;
(SEQ ID NO. 13)
  7) F primer: cagtgcccacgtgtccc;
(SEQ ID NO. 14)
        R primer: gacagccctcctgcattc;
(SEQ ID NO. 15)
  8) F primer: ctttgttcttcacttccccg;
(SEQ ID NO. 16)
        R primer: ctcggtccctgtatccacc;
(SEQ ID NO. 17)
  9) F primer: agctgcttgggatgtaccagc;
(SEQ ID NO. 18)
        R primer: cggcttcctttagtgacttccg;
(SEQ ID NO. 19)
10) F primer: ctagggacctctggggtagc;
(SEQ ID NO. 20)
        R primer: gtgaggggcaggaaagc;
(SEQ ID NO. 21)
11) F primer: aaagtactgggctgtggcag;
(SEQ ID NO. 22)
        R primer: gcaaagccctgaagacaaac;
(SEQ ID NO. 23)
12) F primer: aatttgggtttgcttaggcctc;
(SEQ ID NO. 24)
        R primer: gttccctctgctcccctcaag;
(SEQ ID NO. 25)
13) F primer: aattgtggggaaagggaaag;
(SEQ ID NO. 26)
        R primer: accaccccacagcctctc;
(SEQ ID NO. 27)
14) F primer: catgggttttatgccagtcc;
(SEQ ID NO. 28)
        R primer: gtccctagcatggtttgctg;
(SEQ ID NO. 29)
15) F primer: ccccagagcccagtcttg;
(SEQ ID NO. 30)
        R primer: gtctcctccaacaccaaagg; or
(SEQ ID NO. 31)
16) F primer: gctccgagagtgcaggg;
(SEQ ID NO. 32)
        R primer: gcaggggctgaatggac.
```

The materials and reagents used for performing said PCR amplification include: genomic DNA extracted from a blood sample of patients suspected of suffering from early-onset PD; a pair of primers; a buffer; PCR polymerase; and, deionized water.

The steps and conditions for performing said PCR amplification can be readily determined by people skilled in the art.

| | Nucleotide | Annealing | Annealing |
|---|---|---|---|
| Primer | Sequence | Temp. (°C.) | Time (sec.) |
| 1F | SEQ ID NO.1 | 55 | 30 |
| 1R | SEQ ID NO. 2 | 55 | 30 |
| 2F | SEQ ID NO. 3 | 55 | 30 |
| 2R | SEQ ID NO. 4 | 55 | 30 |
| 3F | SEQ ID NO. 5 | 55 | 30 |
| 3R | SEQ ID NO. 6 | 55 | 30 |
| 4F | SEQ ID NO. 7 | 60 | 30 |
| 4R | SEQ ID NO. 8 | 60 | 30 |
| 5F | SEQ ID NO. 9 | 55 | 30 |
| 5R | SEQ ID NO. 10 | 55 | 30 |
| 6F | SEQ ID NO. 11 | 60 | 30 |
| 6R | SEQ ID NO. 12 | 60 | 30 |
| 7F | SEQ ID NO. 13 | 55 | 30 |
| 7R | SEQ ID NO. 14 | 55 | 30 |
| 8F | SEQ ID NO. 15 | 58 | 30 |
| 8R | SEQ ID NO. 16 | 58 | 30 |
| 9F | SEQ ID NO. 17 | 55 | 30 |
| 9R | SEQ ID NO. 18 | 55 | 30 |
| 10F | SEQ ID NO. 19 | 58 | 30 |
| l0R | SEQ ID NO. 20 | 58 | 30 |
| 11F | SEQ ID NO. 21 | 55 | 30 |
| 11R | SEQ ID NO. 22 | 55 | 30 |
| 12F | SEQ ID NO. 23 | 55 | 30 |
| 12R | SEQ ID NO. 24 | 55 | 30 |
| 13F | SEQ ID NO. 25 | 60 | 30 |
| 13R | SEQ ID NO. 26 | 60 | 30 |
| 14F | SEQ ID NO. 27 | 58 | 30 |
| 14R | SEQ ID NO. 28 | 58 | 30 |
| 15F | SEQ ID NO. 29 | 55 | 30 |
| 15R | SEQ ID NO. 30 | 55 | 30 |
| 16 + 17 F | SEQ ID NO. 31 | 60 | 30 |
| 16 + 17R | SEQ ID NO. 32 | 60 | 30 |

A PLA2G6 gene mutation pattern can be then determined by sequence analysis.

If the PLA2G6 gene mutation pattern comprises a nucleotide mutation of G991T, then the patients can be recognized as ones suffering from early-onset PD.

Accordingly, the present invention discloses a method for diagnosing early-onset PD in a subject, comprising the steps of:

(1) extracting genomic DNA from a blood sample of patients suspected of suffering from early-onset PD;

(2) performing PCR amplification of a coding region of PLA2G6 gene by using a pair of F primer and R primer as shown in Table 1; and

(3) sequencing the DNA fragments obtained from the PCR amplification, and aligning for comparison the sequenced DNA fragments with the correspondent sequenced DNA fragments obtained from a healthy subject,

wherein, if there is nucleotide G991T mutation in the coding region of PLA2G6 gene, then the patients can be recognized as ones suffering from early-onset PD.

Further, the present invention provides a kit of detecting the presence of a nucleotide mutation of G991T in the coding region of PLA2G6 gene for diagnosing early-onset PD in a subject, comprising: a pair of F primer and R primer as shown in Table 1; a buffer; PCR polymerase; and, an instruction for use.

### BRIEF DESCRIPTION OF THE DRAWINGS

FIG. 1. The enzyme activity of PLA2G6 is significantly reduced in the SN of

PLA2G6^{D331Y/D331Y} knockin mice. (A) PCR assays were performed to genotype wild-type mice, PLA2G6^{WT/D331Y} mice and PLA2G6^{D331Y/D331Y} mice. (B) Sanger sequencing of cDNA synthesized from the SN of PLA2G6^{WT/D331Y} or PLA2G6^{D331Y/D331Y} mice verified the (G991→T991) nucleotide mutation at the residue D331 of PLA2G6. As a result, D331 (GAC) was converted to Y331 (TAC). (C) Western blot analysis using anti-PLA2G6 antibody showed that cytosolic or mitochondrial protein level of PLA2G6 in the SN of heterozygous (D331Y) PLA2G6 mice or homozygous (D331Y) PLA2G6 mice was similar to that of PLA2G6 in the SN of wild-type mice. (D) The enzyme activity of PLA2G6 was reduced in the SN of PLA2G6^{WT/D331Y} mice or PLA2G6^{D331Y/D331Y} mice compared to that of wild-type mice. Data are presented as mean±SEM of 20 mice. **P<0.01, ***P<0.001 compared with wild-type mice.

FIG. 2. PLA2G6^{D331Y/D331Y} KI mice display neuronal death of SNpc dopaminergic cells and the loss of nigrostriatal dopaminergic terminals. (A and C) (tyrosine hydroxylase) (TH) immunostaining demonstrated that compared to age-matched WT mice or heterozygous PLA2G6^{WT/D331Y} mice, the number of TH⁺-dopaminergic neurons was significantly reduced in the SNpc of PLA2G6^{D331Y/D331Y} KI mice at the age of 6 or 9 months. Scale bar is 200 μm. Each bar shows the mean±SEM of 20 mice. (D) Six- or nine-month old PLA2G6^{D331Y/D331Y} knockin mice displayed a significantly decrease in the number of SNpc Niss1⁺ cells. Each bar represents the mean±SEM of 20 mice. (B and E) Immunohistochemical staining of NeuN demonstrated the absence of a significant neurodegeneration in the striatum of PLA2G6^{D331Y/D331Y} KI mice. Scale bar is 200 pm. Each bar shows the mean±SEM of 20 mice. (F) The VMAT2 density of striatal dopaminergic nerve terminals was visualized by performing in vivo ¹⁸F-FP-DTBZ microPET imaging. Compared to WT or PLA2G6^{WT/D331Y} mice, striatal ¹⁸F-FP-DTBZ uptake of PLA2G6^{D331Y/D331Y} knockin mice at the age of 6 months or 9 months was significantly decreased. Ear bar represents the mean±SEM of 6 mice. (G) Compared with age-matched WT mice or heterozygous PLA2G6^{WT/D331Y} mice, 9-month-old PLA2G6^{D331Y/D331Y} KI mice displayed a significant decrease in striatal density of TH-immunoreactive staining. Scale bar is 200 μm. Each bar represents as the mean±SEM of 20 mice. *P<0.05, **P<0.01 compared with wild-type mice.

FIG. 3. Lewy body pathology is found in the SN o PLA2G6^{D331Y/D331Y} KI mice at the age of 9 months. (A-F) Lewy body in the SN of PLA2G6^{D331Y/D331Y} KI mice at the age of 9 months was detected by performing immunocytochemical staining of anti-p-αSyn (A-C) and anti-αSyn (D-F). Scale bar is 50 μm. (G) Immunoblotting analysis showed an upregulated protein expression of αSyn or p-αSyn in the SN of homozygous PLA2G6^{D331Y/D331Y} KI mice at the age of 9 months. The same observation was found in other four experiments. (H) The expression of tau protein (Tau) in SN of PLA2G6^{D331Y/D331Y} mice was similar to PLA2G6^{WT/WT} mice Immunoblotting assay demonstrated that an increased protein level of phospho-tau^{Ser202/Thr205} (p-Tau) was found in the SN of homozygous PLA2G6^{D331Y/D331Y} mice at the age of 9 months. The expression level of proteins was quantified by the densitometer. The same observation was found in other four experiments. Each bar shows the mean±SEM value of five independent experiments. *P<0.05, **P<0.01 compared with wild-type mice.

FIG. 4. PLA2G6^{D331Y/D331Y} KI mice display early-onset motor deficits of parkinsonism. The velocity (A) and distance (B) of locomotor activity were progressively decreased in PLA2G6^{D331Y/D331Y} mice at the age of 6 to 12 months. Age-matched heterozygous PLA2G6^{WT/D331Y} mice did not exhibit the phenotype of hypoactivity. Ear bar represents the mean±SEM of 20 mice. (C) Cylinder test was conducted to determine the spontaneous activity of animal. The motor deficit of 6- to 12-month-old homozygous PLA2G6^{D331Y/D331Y} mice was indicated by a significant decrease in the number of rears. Each bar shows the mean±SEM of 20 animals. (D) Rotarod test demonstrated that compared to age-matched WT or PLA2G6^{WT/D331Y} mice, 6- to 12-month-old PLA2G6^{D331Y/D331Y} KI mice displayed a marked reduction in the latency to fall. Each bar represents the mean±SEM of 20 animals. (E) Pole test demonstrated that homozygous PLA2G6^{D331Y/D331Y} mice at the age of 6 to 12 months displayed motor dysfunction by taking a longer time to execute the pole test. Each bar shows the mean±SEM of 20 animals. Forty minutes after injecting saline or methyl L-DOPA (1.5 mg/kg of body weight) into animals, the distance (F) and velocity (G) of locomotion activity were analyzed. PLA2G6^{D331Y/D331Y} mice injected with saline exhibited a reduced distance and velocity of locomotor activity. Following the administration of methyl L-DOPA, the hypoactivity displayed by 9-month-old PLA2G6^{D331Y/D331Y} KI mice was improved, which was indicated by an increase in the locomotor activity (F and G). Each bar shows the mean±SEM of 10 animals. *P<0.05, **P<0.01 compared to WT mice. ^{#}P<0.05 compared to PLA2G6^{D331Y/D331Y} mice injected with saline.

FIG. 5. Homozygous (D331Y) PLA2G6 mutation leads to abnormality in the ultrastructure of mitochondria and mitochondrial dysfunction. (A-C) In the neuromelanin-positive putative SNpc dopaminergic neurons of WT or PLA2G6^{WT/D331Y} mice, mitochondrial morphology was intact, and mitochondrial cristae had a regular arrangement. In contrast, disrupted structure of mitochondria cristae was observed in the neuromelanin organelle-containing putative SNpc dopaminergic neurons of homozygous PLA2G6^{D331Y/D331Y} mice. The arrow indicates neuromelanin organelle. (D) Compared to WT mice, the size of mitochondria was decreased in the SN of homozygous (D331Y) PLA2G6 mice. Each bar shows the mean±SEM of 672-886 mitochondria. (E-H) The activity of mitochondrial complex I and III was significantly decreased in the SN of PLA2G6^{D331Y/D331Y} KI mice as compared to WT mice. The activity of mitochondrial complex II or complex IV in the SN of homozygous (D331Y) PLA2G6 KI mice was not significantly different from that of WT mice.

For mitochondrial complex I experiments, each bar represents the mean±SEM of 20 mice. For mitochondrial complex II-IV experiments, each bar shows the mean±SEM of 10 mice. The activity of mitochondrial complex was normalized with the activity of citrate synthase. (I) Intracellular ATP level was reduced in the SN of homozygous (D331Y) PLA2G6 KI mice. (J) Compared to WT or PLA2G6^{WT/D331Y} mice, overproduction of ROS was observed in the SN of PLA2G6^{D331Y/D331Y} KI mice. (K) The level of mitochondrial lipid peroxidation was upregulated in the SN of homozygous PLA2G6^{D331Y/D331Y} mice. (L) Cytosolic level of cytochrome c was increased in the SN of PLA2G6^{D331Y/D331Y} mice. **P<0.01 compared with wild-type mice. Each bar represents the mean±SEM of 20 animals.

FIG. 6. Homozygous (D331Y) PLA2G6 mutation causes activation of mitochondria apoptotic cascade, induction of ER stress and mitophagy dysfunction. (A) Western blot analysis showed that protein expression of active caspase-3, active caspase-9 or cytochrome c (Cyto c) in the cytosol was upregulated in the SN of homozygous (D331Y) PLA2G6 KI mice at the age of 9 months. (B) Protein levels of Grp78, IRE1, PERK and CHOP were increased in the substantia nigra of PLA2G6^{D331Y/D331Y} knockin mice. (C) Immunoblotting analysis showed that protein expression of parkin or BNIP3 was significantly downregulated in the SN of PLA2G6^{D331Y/D331Y} KI mice. *P<0.05, **P<0.01 compared with WT mice. Each bar shows the mean±SEM of five experiments.

FIG. 7. Homozygous (D331Y) PLA2G6 KI mice display differential gene expressions. (A) Heat maps with hierarchical clustering revealed dramatic gene dysregulation in the substantia nigra of PLA2G6^{D331Y/D331Y} KI mice at the age of 9 months compared with WT mice at the same age. (B) Quantitative real-time PCR analysis was performed to validate selected ten genes. The mRNA levels of Bmp6, Ccnd2, Ctnnb1, Hspa1b, Kidin220, Mapk1, Psap and Sdc2 were decreased in the substantia nigra of PLA2G6^{D331Y/D331Y} mice at the age of 9 months. The mRNA expression of Mark4 or Xaf1 was upregulated in the SN of PLA2G6^{D331Y/D331Y} KI mice. Each bar represents the mean±SEM of four experiments. (C) Compared to WT mice, mRNA levels of eight genes (Bmp6, Ccnd2, Hspa1b, Kidin220, Mapk1, Psap and Sdc2) were decreased in the SN of 5-month-old PLA2G6^{D331Y/D331Y} mice. The mRNA expression of Mark4 or Xaf1 was upregulated in the substantia nigra of PLA2G6^{D331Y/D331Y} mice at the age of 5 months. Each bar shows the mean±SEM of four independent experiments. (D and E) Protein levels of BMP6, CCND2, CTNNB1, HSPA1B, KIDINS220, MAPK1, PSAP and SDC2 were decreased in the substantia nigra of PLA2G6^{D331Y/D331Y} mice. Protein levels of MARK4 and XAF1 were increased in the substantia nigra of homozygous (D331Y) PLA2G6 mice at the age of 9 months. *P<0.05, **P<0.01 compared with wild-type mice. Each bar shows the mean±SEM of four experiments.

FIG. 8. Detection of nucleotide mutation at position 991 from G to T in PLA2G6 gene was performed by (A) PCR amplification and (B) sequence analysis.

### EXAMPLES

### Generation of PLA2G6^{D331Y/D331Y} Knockin (KI) Mice

PCR-based site-directed mutagenesis was conducted to alter the codon GAC for Asp-331 located in the exon 7 of mouse PLA2G6 gene into TAC encoding Tyr. Then, exon 7 fragment with (D331Y) mutation was subcloned into pBluescript SK vector. For the knockin target vector of PLA2G6^{D331Y}, a neomycin selection cassette flanked by LoxP sites was inserted at site of 401 nucleotides downstream the start of exon 7. To target the exon 7, 4.9 kb fragment upstream of exon 7 and 2.2 kb fragment downstream of neomycin selection cassette were obtained and functioned as 5′ and 3′ homologous arms, respectively. Subsequently, knockin targeting fragments of PLA2G6^{D331Y} were subcloned into pBluescript vector containing mutated exon 7 fragment and LoxP-flanked neomycin selection cassette.

The 129/Sv embryonic stem (ES) cells were transfected with XhoI-linearized knockin target vector of PLA2G6^{D331Y}. PCR assays were performed to screen neomycin-resistant colonies with correct homologous recombination. Chimeric mice were obtained by microinjecting correctly targeted ES clone into C57BL/6J blastocysts. F1 heterozygous mutant mice were bred from wild-type C57BL/6J mice and chimeric mice. To remove Neo cassette, F1 mice with germline transmission of (D331Y) PLA2G6 knockin allele were bred with Cre deleter transgenic mice, which express Cre recombinase in the whole body. Then, stable lines of mutant (D331Y) PLA2G6 knockin mice were established by mating F2 PLA2G6^{WT/D331Y} mice with wild-type C57BL/6J mice. The resultant heterozygous knockin mice were bred and maintained on C57BL/6J genetic background and intercrossed to generate homozygous PLA2G6^{D331Y/D331Y} knockin mice. Animal experiments were performed in accordance with protocols approved by Institutional Animal Care and Use Committee (IACUC) of Chang Gung University.

### Subcellular Fractionation

Cytosolic and mitochondrial fractions of SN dissected from WT or KI mice were prepared. Briefly, SN tissues were homogenized in ice-cold buffer comprising 10 mM HEPES (pH7.3), 1 mM DTT, 1 mM EGTA, 70 mM sucrose, 210 mM mannitol and commercial protease inhibitor cocktail (Sigma). Cell lysate was centrifuged at 500×g for 10 min at 4° C. Then, the supernatant was collected and centrifuged at 9500×g for 10 min at 4° C. to obtain the pellet, which was mitochondrial fraction. The supernatant was further centrifuged at 16,000×g for 20 min at 4° C. to obtain cytosolic fraction.

### Western Blot

Mitochondrial or cytosolic proteins (30 μg) were separated on a 12% or 15% SDS-polyacrylamide gel and then transferred onto nitrocellulose membranes. Subsequently, membranes were incubated overnight at 4° C. with following primary antibodies: (1) Monoclonal anti-PLA2G6 antibody (Santa Cruz Biotechnology, sc-376563); (2) Polyclonal anti-phospho-alpha synuclein^{Ser129} antiserum (Abcam, ab51253); (3) Polyclonal anti-alpha synuclein antibody (Proteintech, #10842-1-AP); (4) Monoclonal anti-Tau antiserum (Santa Cruz Biotechnology, sc-32274); (5) Monoclonal anti-phospho-Tau (Ser202, Thr205) antibody (Thermo Scientific, AT8); (6) Polyclonal anti-BiP/Grp78 antiserum (Cell Signaling Technology, #3177); (7) Polyclonal anti-CHOP antibody (Cell Signaling Technology, #2895); (8) Polyclonal anti-IRE 1 a antiserum (Cell Signaling Technology, #3294); (9) Polyclonal anti-PERK antibody (Cell Signaling Technology, #5683); (10) Polyclonal anti-LC3A/B antiserum (Cell Signaling Technology, #12741); (11) Polyclonal anti-cytochrome c antibody (Abcam, ab133504); (12) Polyclonal anti-cleaved caspase-9 antiserum (Cell Signaling Technology, #20750); (13) Polyclonal anti-cleaved caspase-3 antibody (Cell Signaling Technology, #9662); (14) Monoclonal anti-BMP6 monoclonal antiserum (Santa Cruz Biotechnology, sc-57042); (15) Monoclonal anti-CCND2 antibody (Thermo Fisher Scientific, clone DCS3.1); (16) Monoclonal anti-CTNNB1 antiserum (Millipore, clone 8E7); (17) Polyclonal anti-GDNF antibody (Abcam, ab18956); (18) Polyclonal anti-HSPA1B antiserum (Sigma, SAB1403949); (19) Polyclonal anti-KIDINS220 antibody (Abcam, ab34790); (20) Polyclonal anti-MAPK1 antiserum (Santa Cruz Biotechnology, sc-292838); (21) Polyclonal anti-MARK4 antibody (Cell Signaling Technology, #4834); (22) Polyclonal anti-PSAP antiserum (GeneTex, GTX101064); (23) Monoclonal anti-SDC2 antibody (Santa Cruz Biotechnology, sc-376160); (24) Polyclonal anti-XAF1 antiserum (GeneTex, GTX51339); (25) Monoclonal anti-parkin antibody (Cell Signaling Technology, #4211); (26) Monoclonal anti-BNIP3 antiserum (Cell Signaling Technology, #44060). Membranes were incubated with appropriate horseradish peroxidase-conjugated secondary antibodies. Then, chemiluminescence kit (Millipore) was used to visualize immunoreactive proteins. The density of gel bands was determined with a densitometer and normalized by actin signals.

### Measurement of PLA2G6 Enzyme Activity

The modified cPLA2 assay kit (Cayman Chemicals, Cat. 765021) was used to analyze the phospholipase A2 activity of PLA2G6 in the SN tissues. According to the stereotaxic atlas of mouse brain, SN was dissected out from coronal midbrain brain slices (500 μm) under a microscope. Then, SN tissues were lysed in a modified Ca²⁺-depleted lysis buffer containing 50 mM HEPES (pH 7.4), 0.1% CHAPS, 4 mM EDTA and commercial protease inhibitor cocktail (Sigma). The substrate arachidonoyl thio-phosphatidylcholine was incubated with the protein sample in a modified Ca²⁺-depleted lysis buffer (2 mg/mL bovine serum albumin, 8 mM Triton X-100, 160 mM HEPES (pH 7.4), 300 mM NaCl, 4 mM EGTA, 60% glycerol and 8 mM Triton X-100) for 60 min at room temperature. Arachidonoyl thio-phosphatidylcholine was hydrolyzed by iPLA₂ and produced free thiols, which were detected by 5,5 o-dithiobis, 2-nitrobenzoic acid. The absorbance at 414 nm was measured and used to calculate the activity of iPLA₂.

### Immunohistochemical Staining

Animals were anesthetized and intracardially perfused with 4% paraformaldehyde in 0.01 M PBS. Then, cryostat sectioned brain slices were incubated with anti-phospho-α-synuclein^{Ser129} polyclonal antibody, monoclonal anti-α-synuclein antibody, anti-NeuN monoclonal antibody or monoclonal anti-tyrosine hydroxylase antibody. After the washes, brain slices were incubated with biotinylated secondary antibody (Vector Laboratories) and then incubated with streptavidin peroxidase conjugates. Subsequently, brain sections were visualized by incubating with diaminobenzidine (Vector Laboratories). The Stereo Investigator software (MBF Biosciences) was used to calculate the number of NeuN⁺-neurons or TH⁺-dopaminergic neurons. ImageJ software (National Institutes of Health) was used to quantify the striatal density of TH⁺ staining.

Animal Image Study with ¹⁸F-FP-DTBZ microPET

Animal imaging study utilizing a positron emission tomography (PET) imaging was acquired by an Inveon preclinical small animal PET scanner (Siemens Medical Solutions) at Molecular Imaging Center of Chang Gung Memorial Hospital. After receiving a single bolus injection of ¹⁸F-FP-DTBZ (12.9±1.47 MBq in 0.1 ml saline), mice were anesthetized with isoflurane, and images were acquired in 3D mode for 90 min. PET images were reconstructed using two-dimensional ordered-subset expectation maximum method without corrections for attenuation, randomness and scatter. Imaging data were processed and analyzed with PMOD software (PMOD Technologies, version 3.2). PET images were co-registered to the corresponding MRI imaging. Regions of interest were drawn within the striatum and cerebellum. Cerebellum was used for the reference region. The striatal specific uptake ratio (SUVR) was calculated as (the uptake of right of left striatum)/(the uptake of cerebellum).

Behavioral Assessments The locomotor activity of WT or (D331Y) PLA2G6 KI mice were evaluated in open field boxes for 60 minutes. Movements of animal were recorded and assessed with the TopScan video tracking system (Clever Sys., Inc.). The locomotor activity of mouse was examined by measuring the velocity and distance. To evaluate the therapeutic benefit of L-DOPA in PLA2G6^{D331Y/D331Y} mice, benserazide (0.5 mg/kg) and L-DOPA (1.5 mg/kg) were intraperitoneally administered into animal. The locomotor activity was recorded 40 min after the injection.

The motor performance of animal was assessed by the pole test. Mouse was placed on top of the pole, and the base of pole was positioned in the home cage. Animals oriented themselves downward and descended along the pole back into home cage. Animals received two consecutive days of training consisting of five trials for each session. On the day of pole test, animals carried out five trials. Motor performance of animal was determined by measuring the time needed for orienting downward and descending.

Rotarod test using an accelerating rotarod apparatus (Ugo Basile Biological Research Apparatus) was conducted to assess motor coordination and balance of animal. Briefly, mouse was placed on the accelerated rotarod, and the latency to fall off the rod was recorded.

Spontaneous movements of animals were analyzed by performing cylinder test. Animals were placed into an acrylic cylinder (12.7 cm diameter; 15.5 cm height), and the number of spontaneous rearing movements was recorded for 3 min

### Transmission Electron Microscopy Study

Mouse was intracardially perfused with 4% paraformaldehyde/2.5% glutaraldehyde fixative. The SN region of brain tissue was cut into 1-mm³ slices by using a vibratome and post-fixed in 1% osmium tetroxide for two hours. After 0.1 M phosphate buffer washing, SN slices were dehydrated through an ascending series of ethanol solutions and embedded in Epon resin (Electron Microscopy Sciences). Following sectioning, ultrathin (80 nm) sections were visualized using transmission electron microscope (JEM-1230, JEOL). The diameters of mitochondria were measured by using ImageJ software.

### Determination of Intracellular ATP Content

Luminescent ATP determination kit (Thermo Fisher Scientific, Cat. A22066) was used to measure cellular level of ATP. Cell lysates (20 μl) were loaded in microplate and incubated with reaction buffer. Luminescence was determined by measuring the absorbance at 560 nm using TECAN luminescence reader (TECAN Infinite M200 Pro).

### Measurement of Mitochondrial Complex I, Complex II, Complex III or Complex IV Activity

The complex I enzyme activity assay kit (Abcam) was used to analyze the complex I activity of mitochondria. Mitochondrial extracts (50 μg) were added and incubated in microplate containing monoclonal anti-complex I antibody. The complex I activity was determined by measuring the oxidation of reduced NADH to NAD⁺, which results in an increased absorbance at 450 nm.

The activity of mitochondrial complex II was determined by using complex II enzyme activity microplate assay kit (Abcam) following the manufacturer's instructions. Briefly, complex II of mitochondrial sample was immunocaptured within the microplate well, which was coated with an anti-complex II monoclonal antibody. Succinate was used as a substrate during performing this assay. The formation of ubiquinol by complex II caused the reduction of the dye DCPIP (2,6-diclorophenolindophenol), and a decrease in the absorbance of DCPIP at 600 nm was detected spectrophotometrically.

Mitochondrial complex III activity was determined by using mitochondrial complex III activity assay kit (BioVision) according to the manufacturer's instructions. Briefly, standard curve for various amounts of reduced cytochrome c was constructed by measuring the absorbance at 550 nm. Mitochondrial samples were added to reaction mixture with or without antimycin A. Following the addition of substrate cytochrome c, the absorbance at 550 nm was measured. The activity of complex III was determined by comparing OD values of samples with the standard curve of reduced cytochrome c.

The enzyme activity of mitochondrial complex IV was measured by using complex IV rodent enzyme activity microplate assay kit (Abcam) following the manufacturer's instructions. Briefly, complex IV of mitochondrial extract was immunocaptured within the sample well of microplate. Following the oxidation of reduced cytochrome c, the activity of complex IV was measured colorimetrically by the change of absorbance at 550 nm.

### Determination of Reactive Oxygen Species (ROS)

OxiSelect In Vitro ROS/RNS assay kit (Cell Biolabs) was used to determine the level of ROS. Mitochondrial extracts (50 μl) were loaded into microplate and incubated with 50 μl of the catalyst reagent, which accelerates the oxidative reaction. Following the incubation for 5 minutes, specific ROS probe DCFH-DiOxyQ was added into the mixture. DCFH-DiOxyQ oxidized by ROS generated fluorescent product dichlorofluorescein (DCF). Fluorescence intensity of DCF was measured with TECAN Infinite M200 Pro microplate reader.

### Analysis of Mitochondrial Lipid Peroxidation

Thiobarbituric acid reactive substances (TBARS) assay kit (Cayman Chemicals, Cat. 10009055) was used to examine the level of mitochondrial lipid peroxidation by quantifying the amount of malondialdehyde (MDA)-thiobarbituric acid (TBA) adduct. The formation of MDA-TBA adduct occurred under acidic conditions and high temperature. MDA standards or mitochondrial samples interacted with TBA at 100° C. for 60 minutes. The amounts of MDA-TBA adducts were then quantified by measuring optical density at 540 nm.

### Measurement of Cytochrome c Release

Cytochrome c ELISA Assay Kit (Thermo Fisher Scientific, Cat. KHO1051) was used to evaluate the release of cytochrome c. Mitochondrial or cytosolic lysates were added into microplate containing monoclonal anti-cytochrome c antibody, followed by adding biotin conjugates. After incubation with streptavidin-HRP working solution, the substrate tetramethylbenzidine was loaded into microplate. Then, the optical density at 450 nm was measured.

### Microarray Study

Total RNA was purified from substantia nigra of wild-type or PLA2G6^{D331Y/D331Y} KI mice using RNeasy Mini Kit (Qiagen). Affymetrix Mouse Genome arrays were used to analyze gene expression profile. Eight samples (wild-type mice, n=4; PLA2G6^{D331Y/D331Y} KI mice, n=4) were prepared and analyzed. Differentially expressed genes were analyzed using the dCHIP software. Differential gene expression was estimated by the mean (PLA2G6^{D331Y/D331Y}/WT) expression ratio.

The selection criteria for significantly altered gene expressions were p<0.05 and fold-change≥1.9.

### Real-Time Quantitative RT-PCR Assay

Quantitative real-time RT-PCR was performed on StepOne Real-Time PCR system (Applied Biosystems). The mRNA level of genes was normalized to reference gene GAPDH. The 2^{-(ΔΔCt)} equation was used to calculate the relative change of mRNA expression.

### Statistics

Data were expressed as the mean±SEM. One-way ANOVA followed by post-hoc Tukey's multiple comparison test was used to analyze statistical significance among multiple groups. Significant difference between two groups was determined by unpaired Student's t-test (two-tailed). A P value of less than 0.05 was considered significant.

### Example 1

### PLA2G6^{D331Y/D331Y} Homozygous Knockin (KI) Mice Exhibit a Decreased PLA2G6 Activity in the Substantia Nigra (SN).

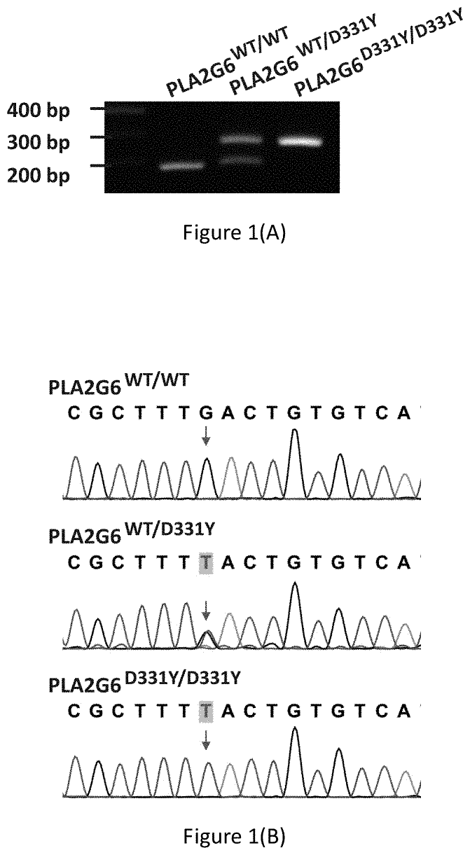

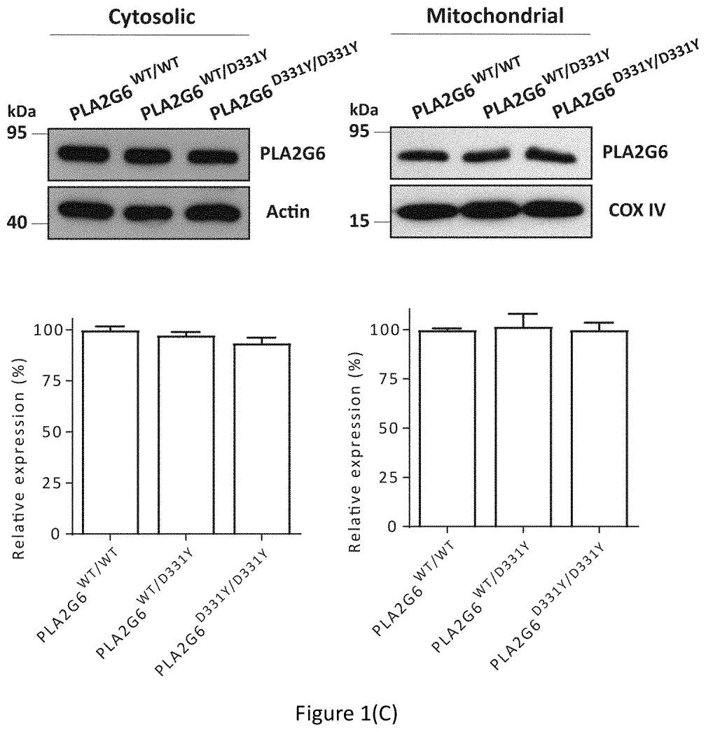

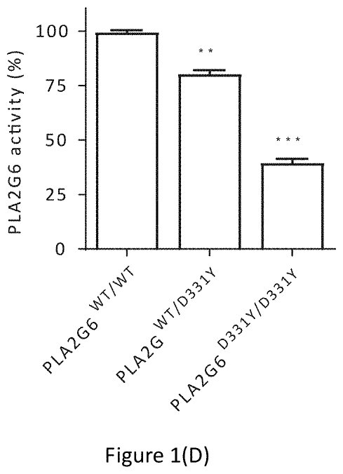

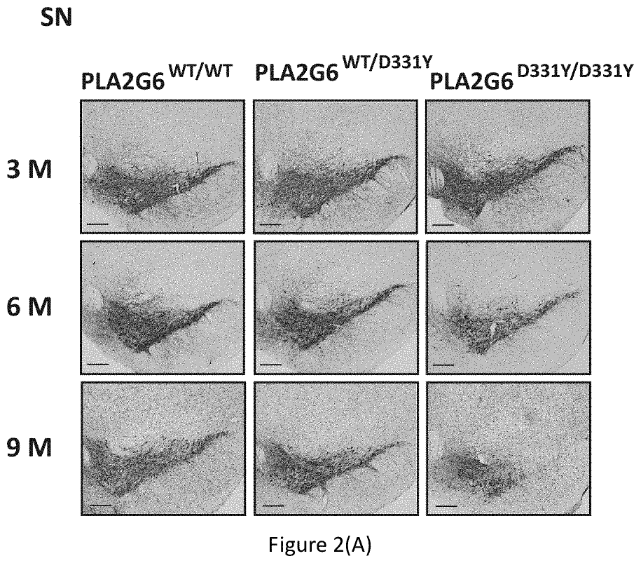

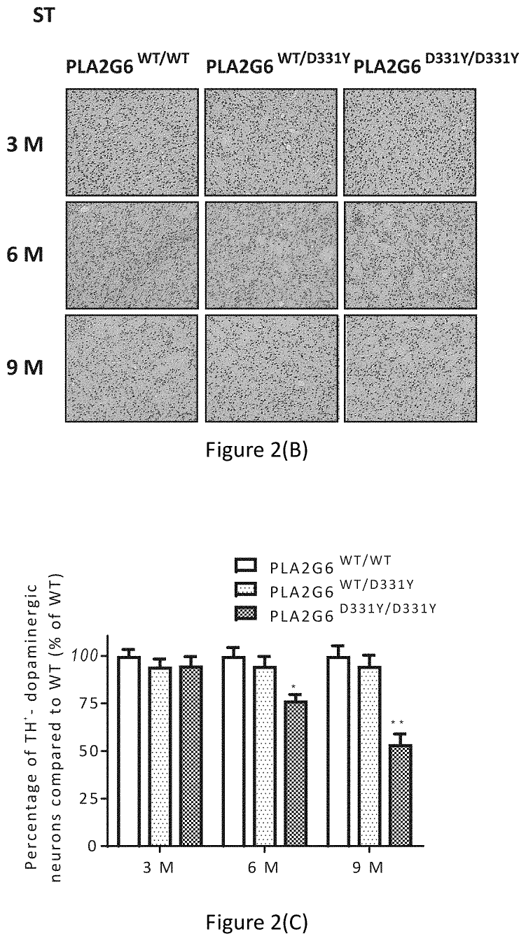

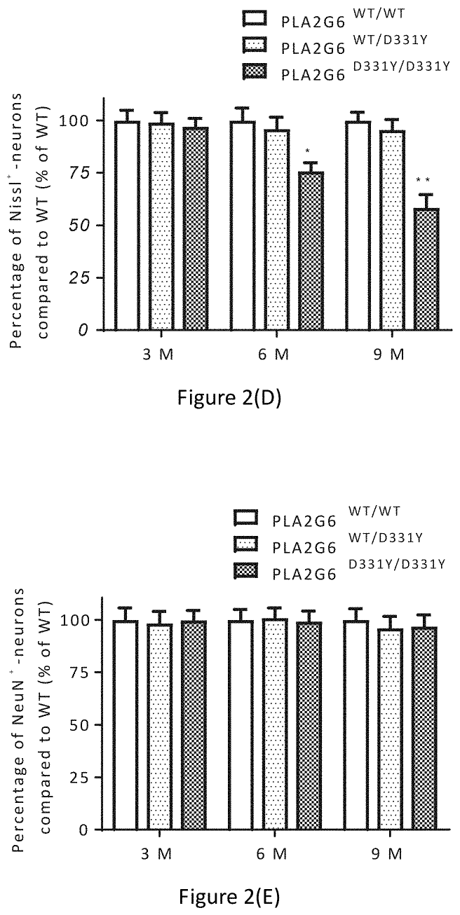

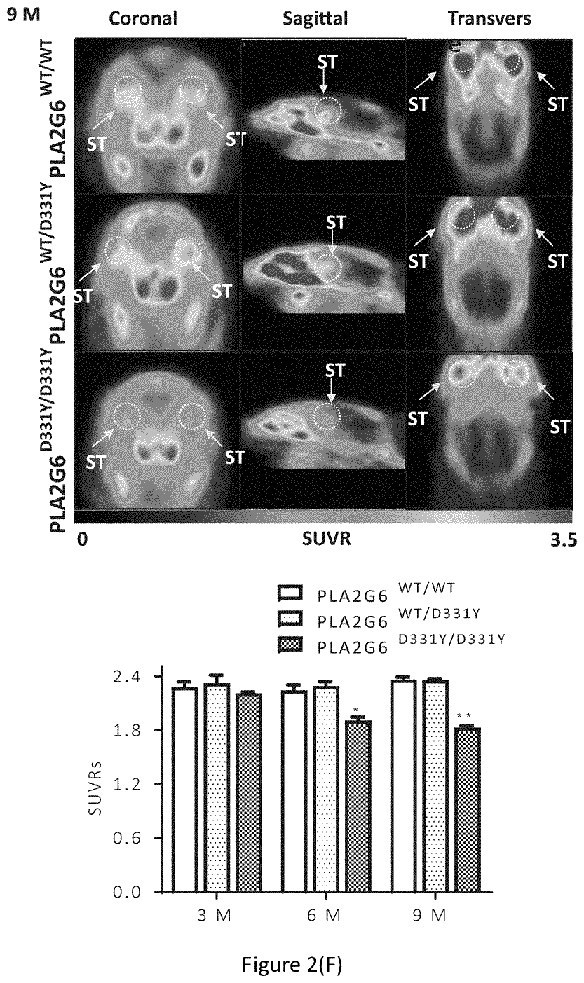

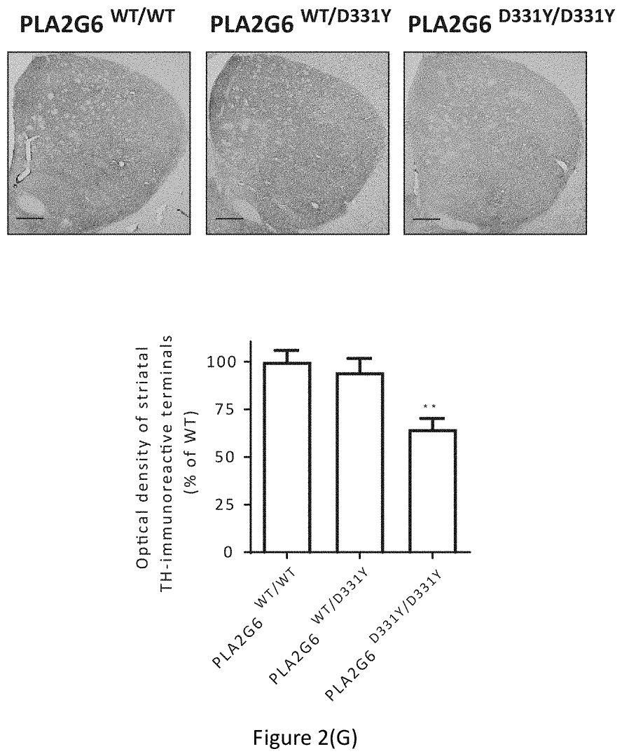

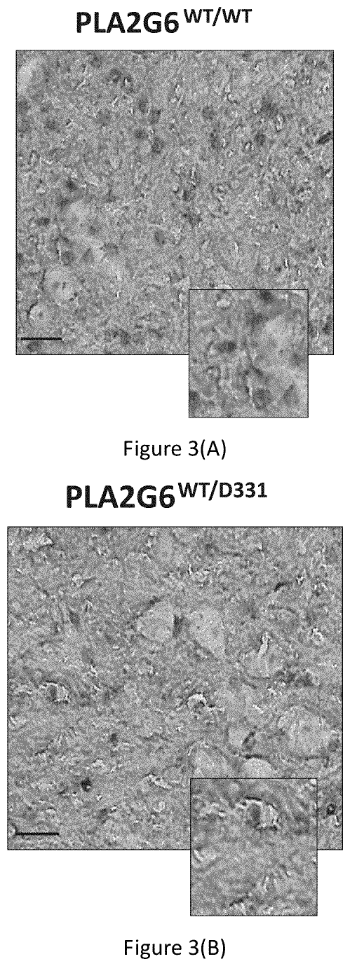

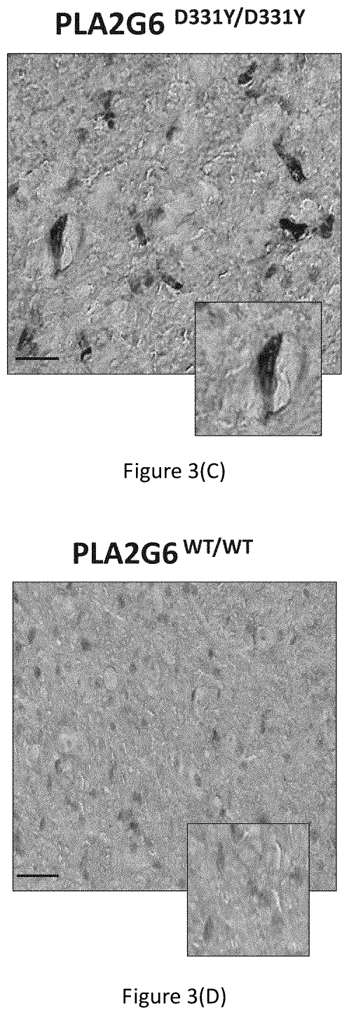

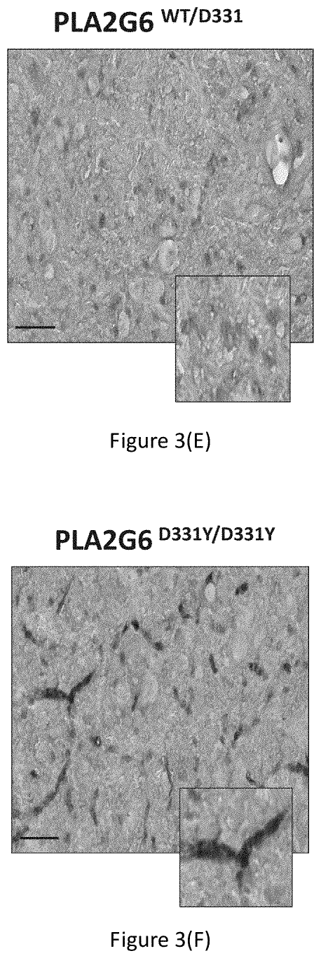

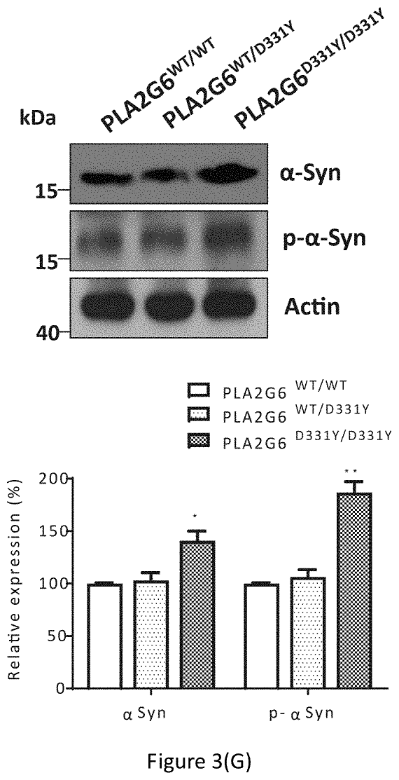

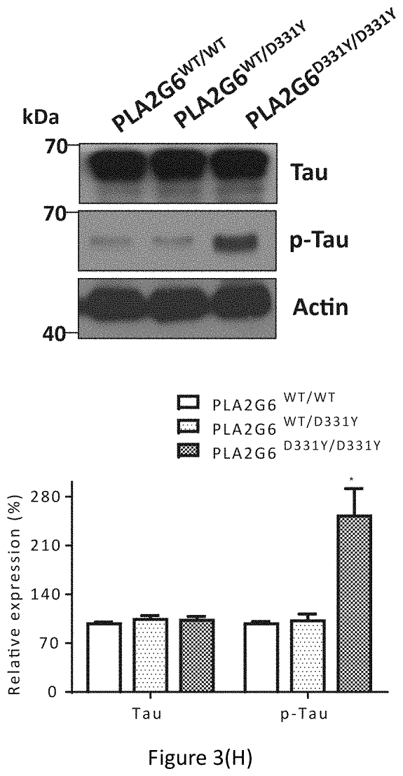

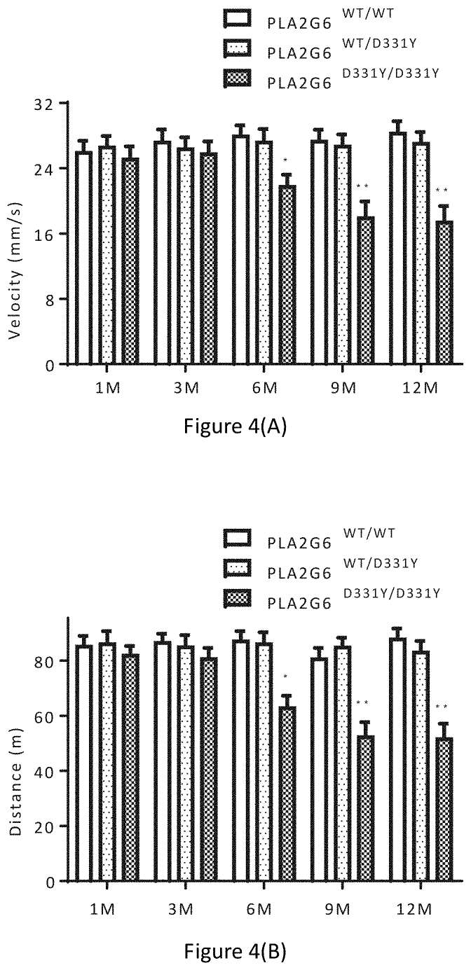

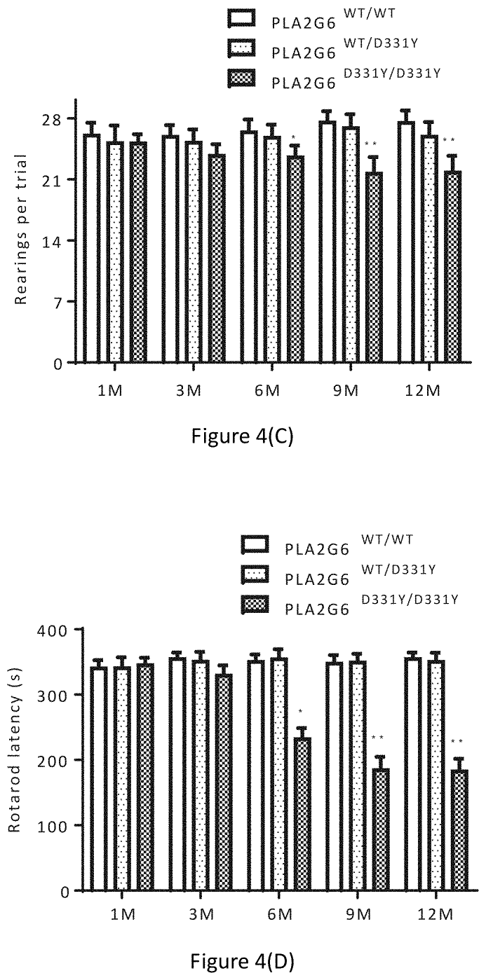

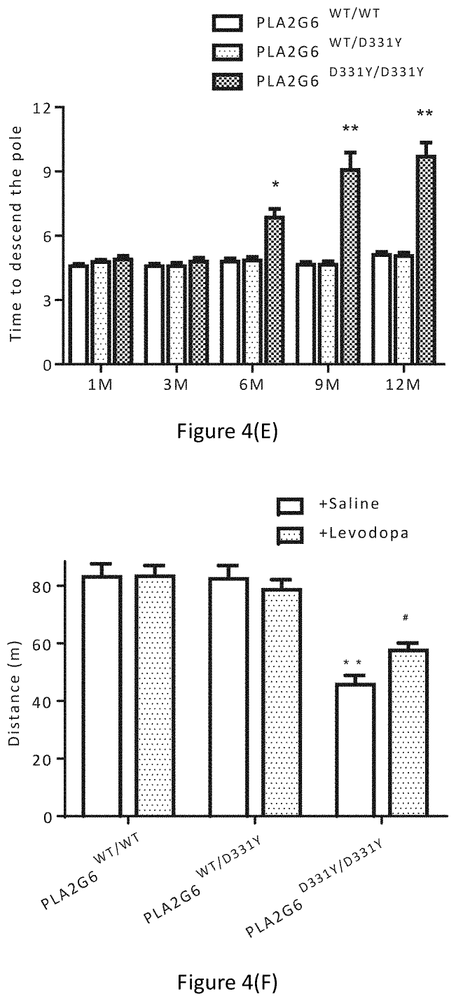

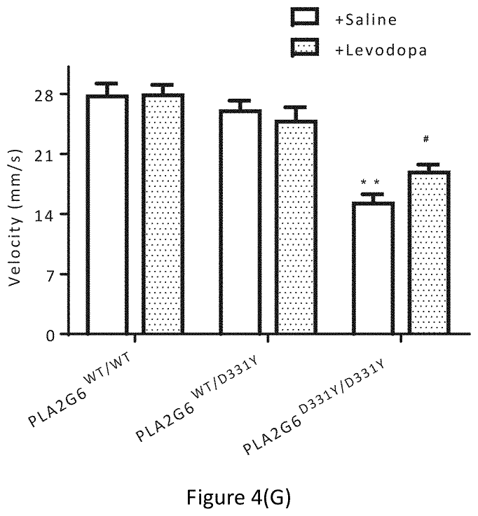

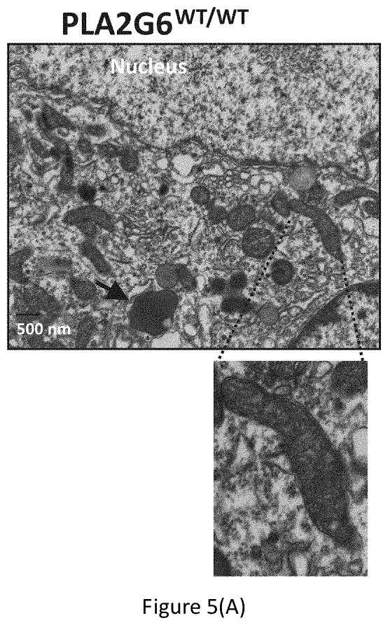

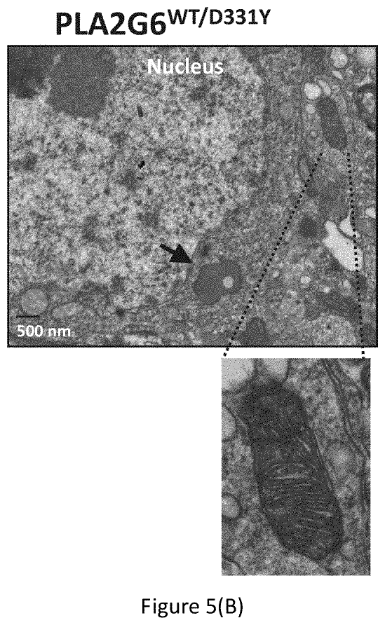

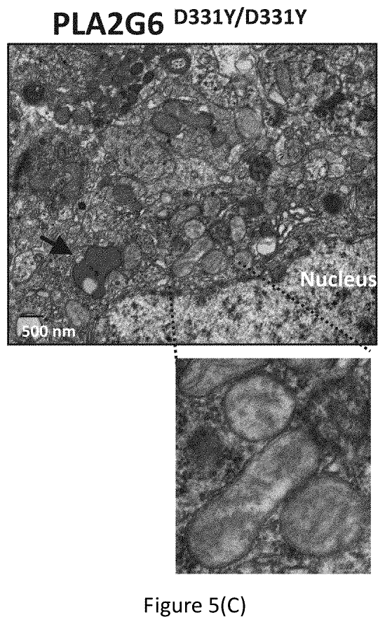

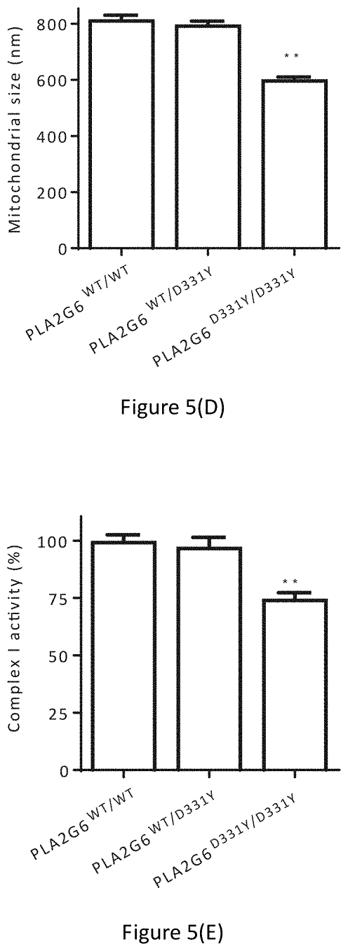

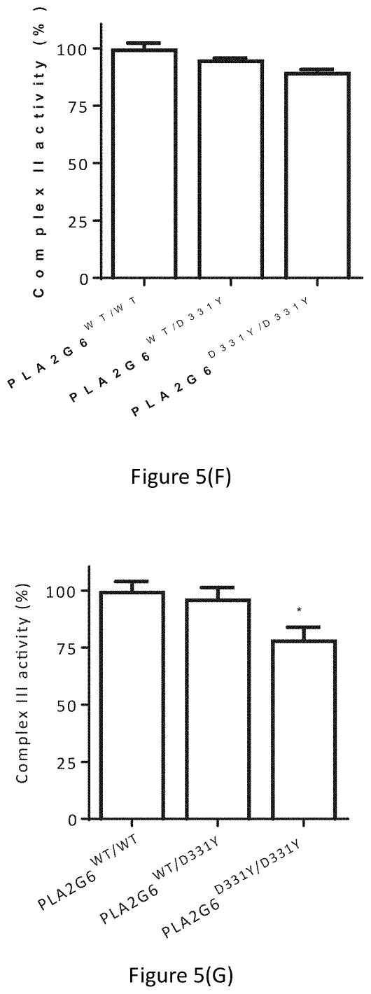

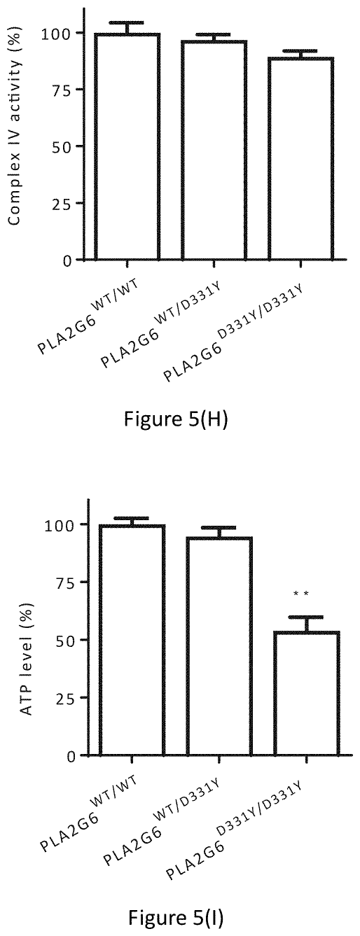

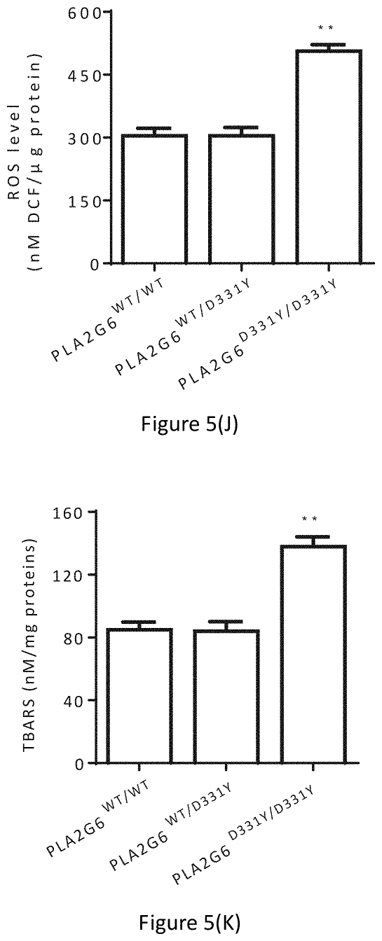

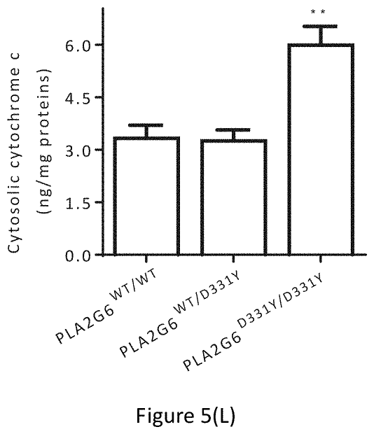

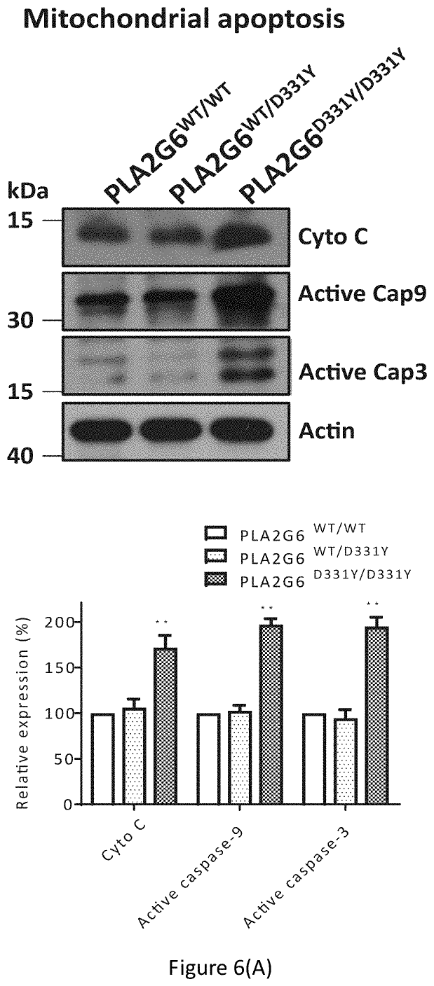

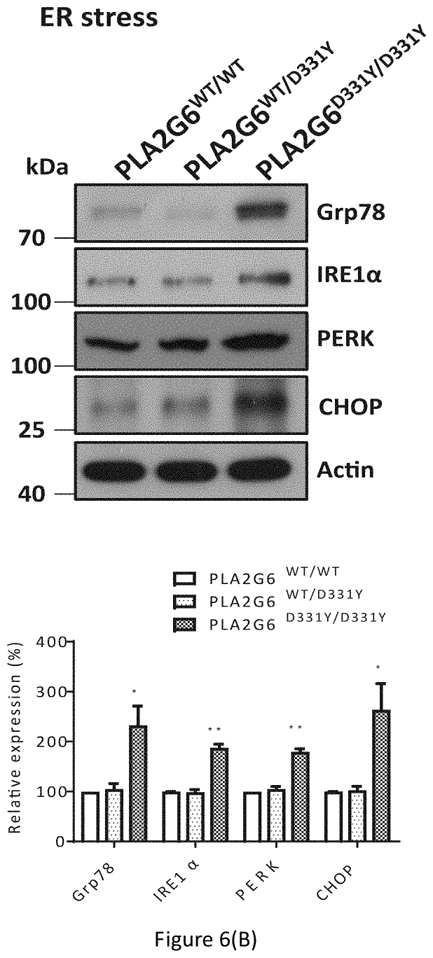

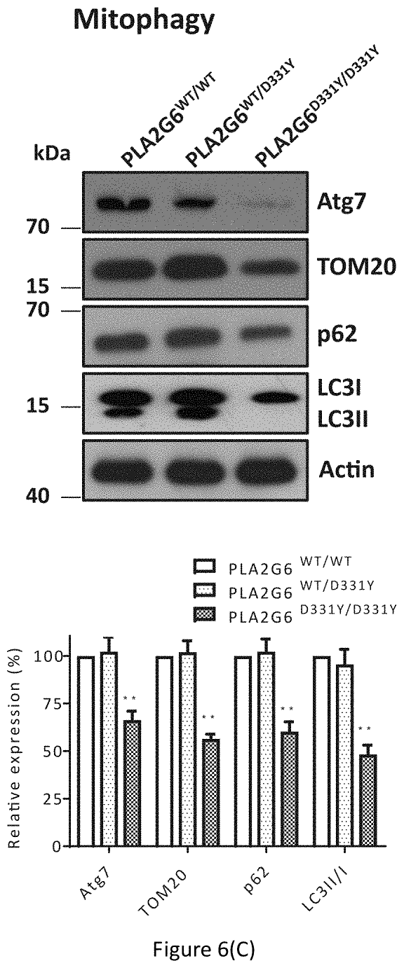

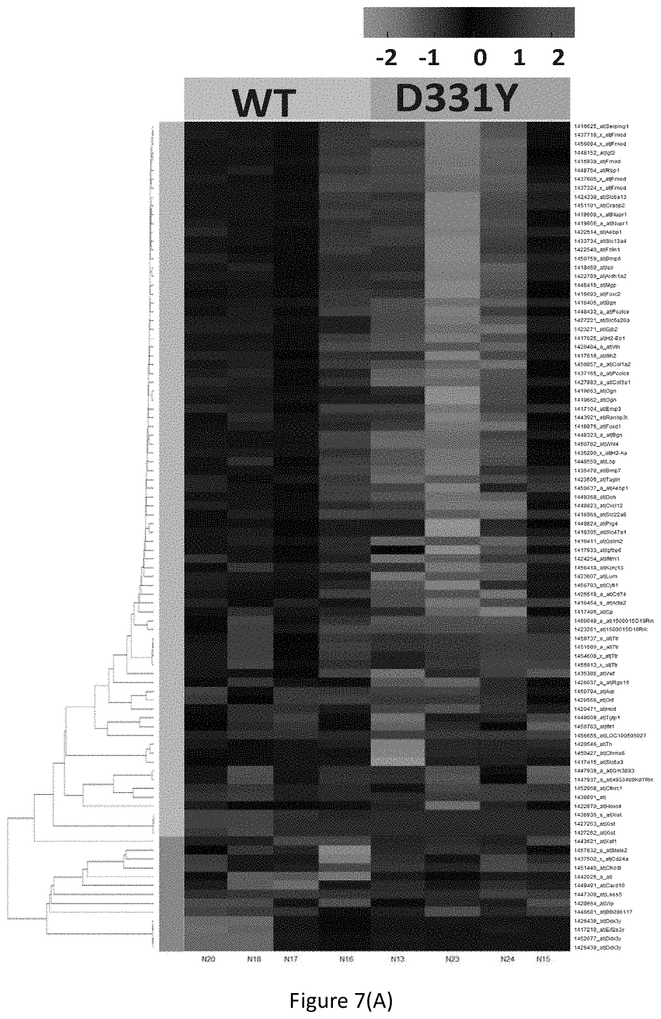

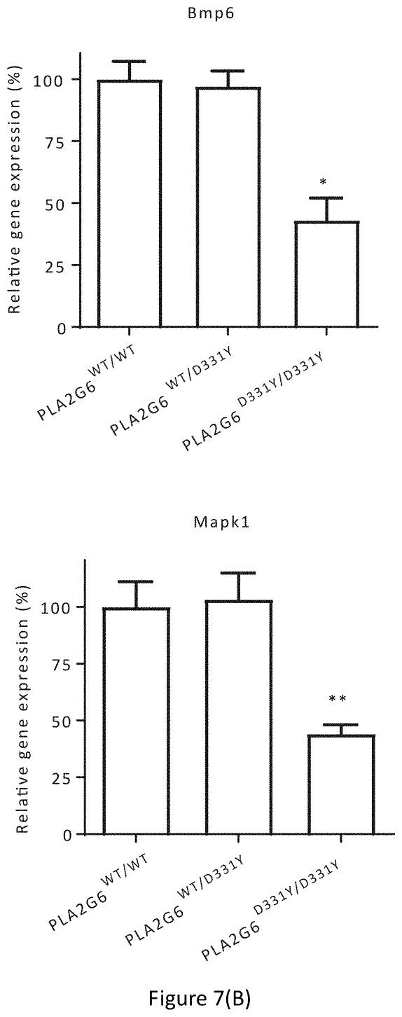

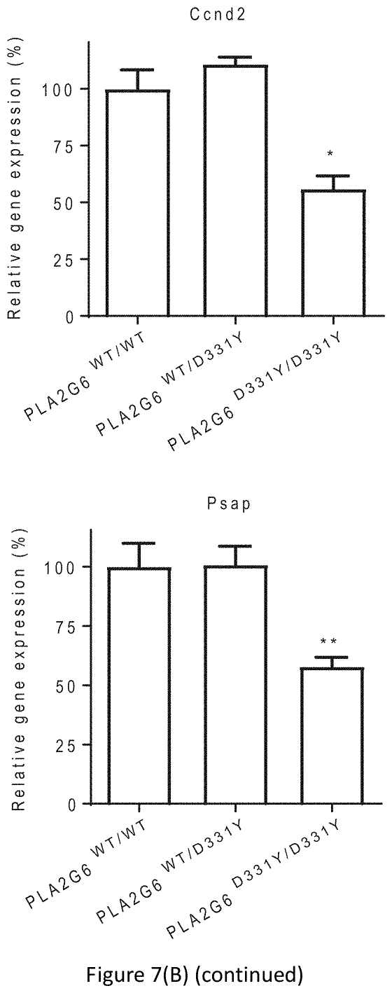

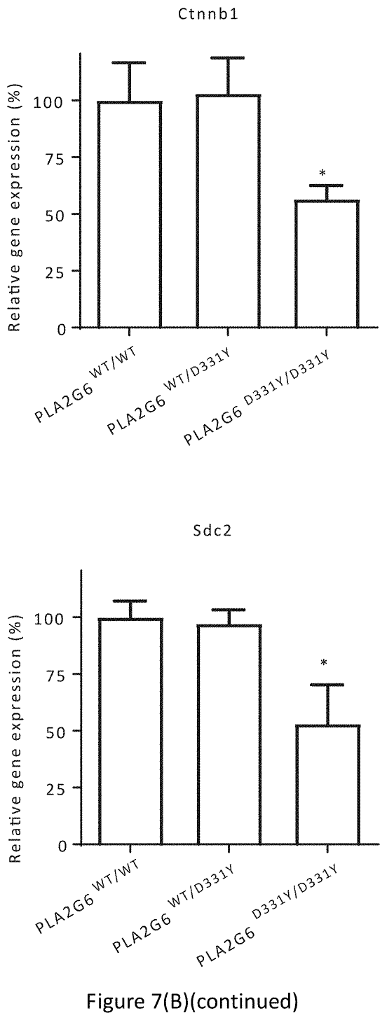

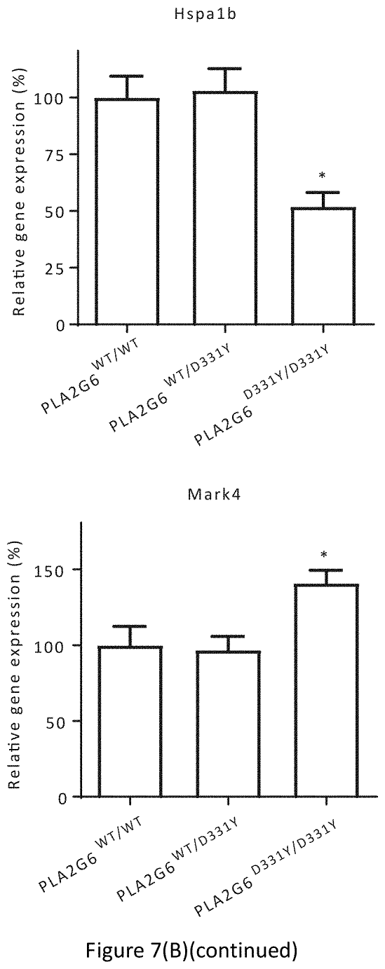

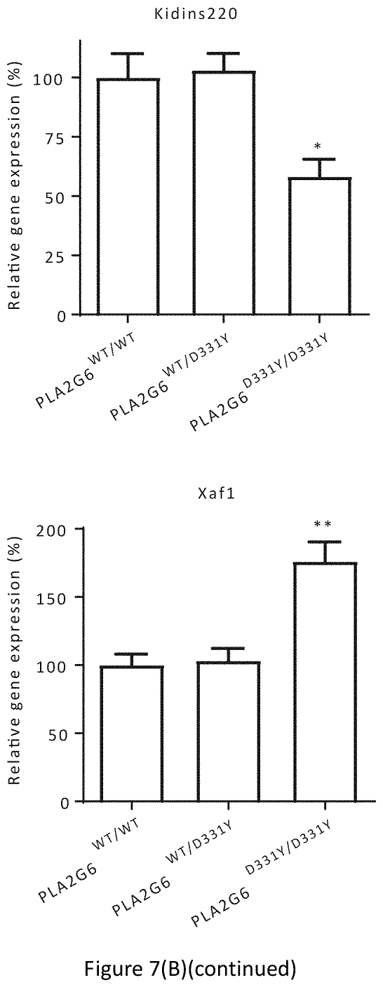

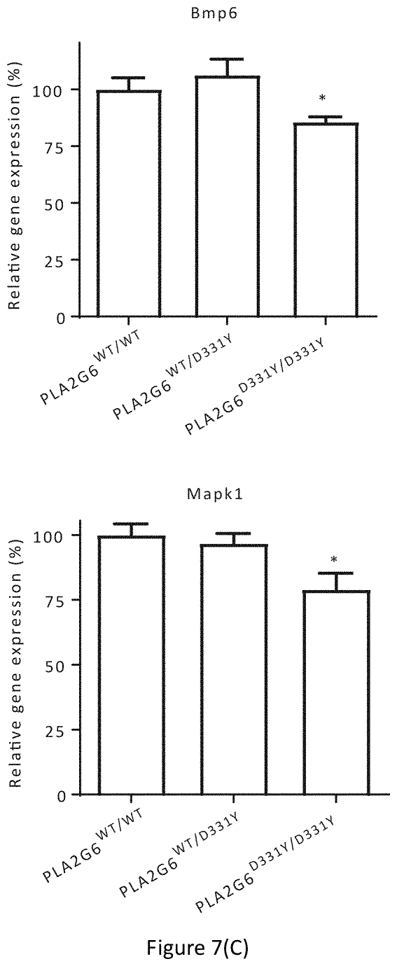

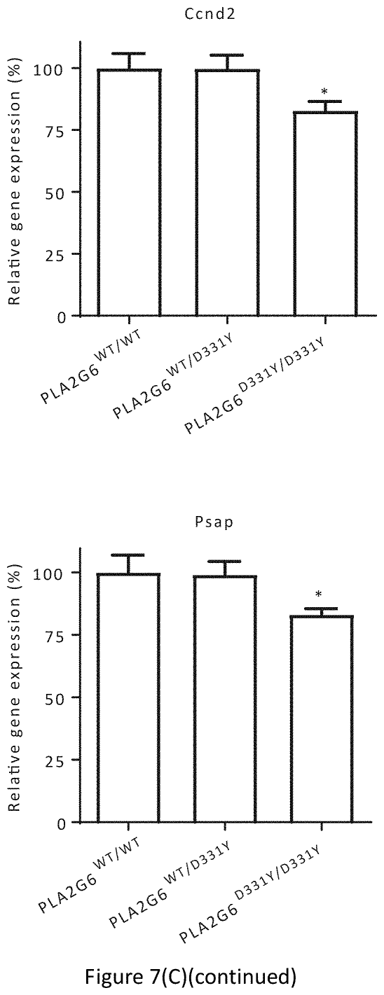

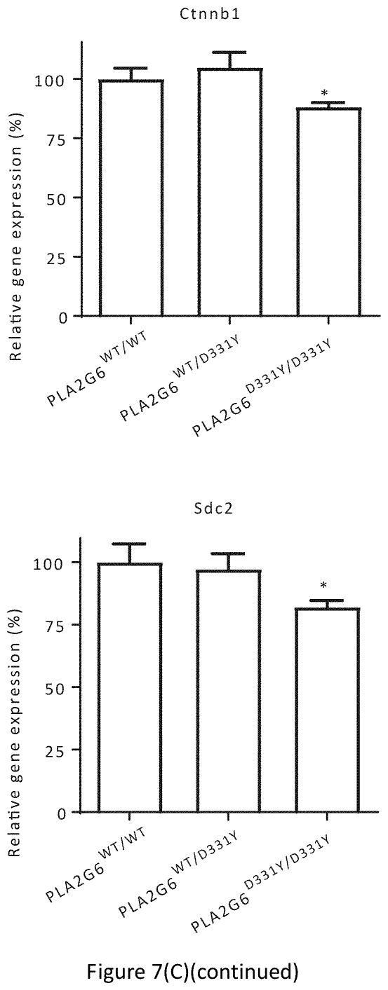

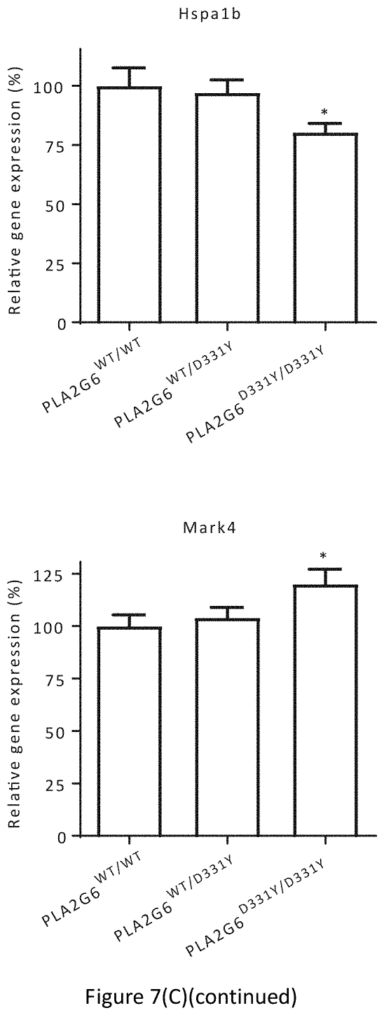

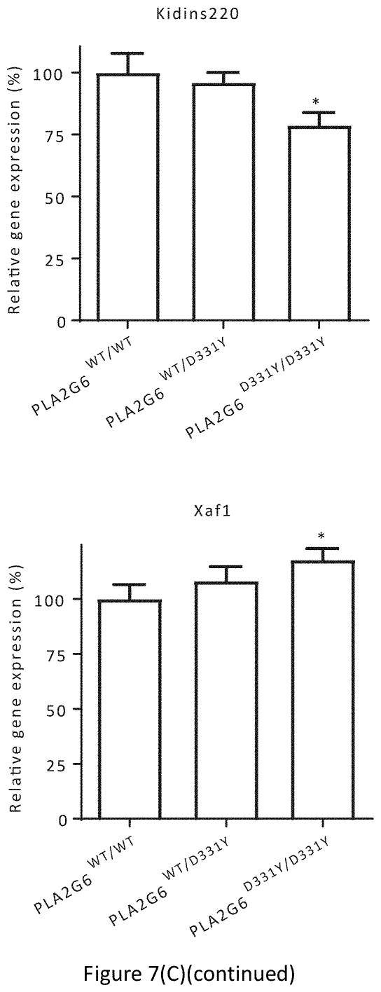

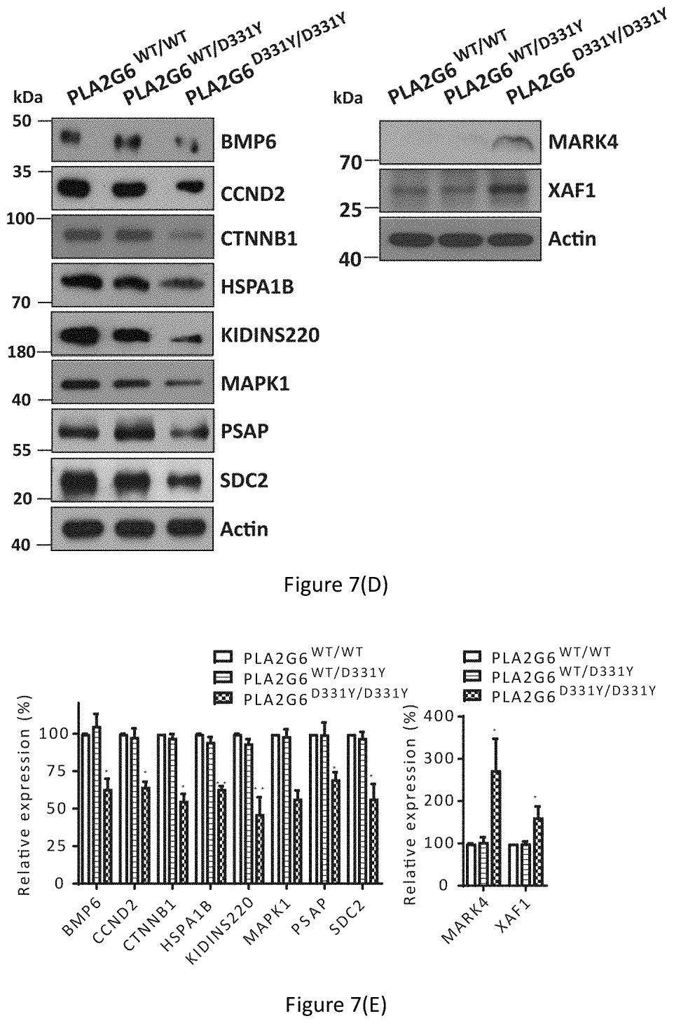

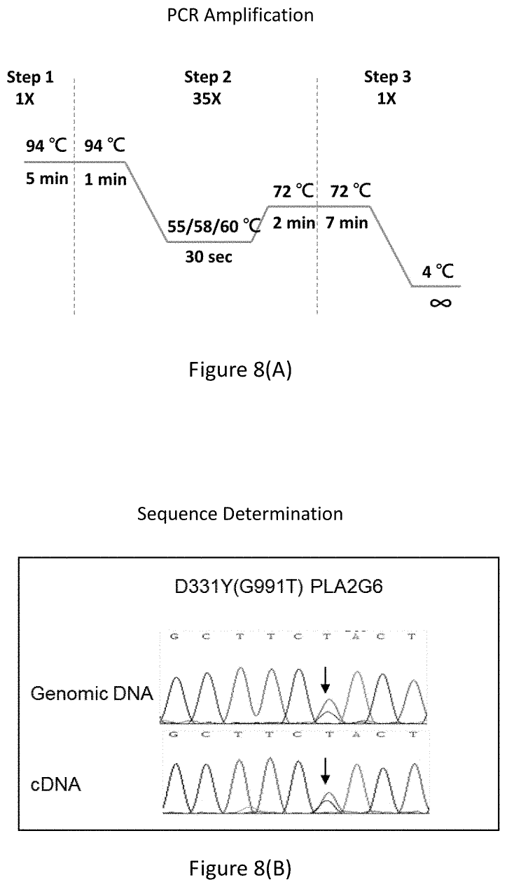

To generate knockin mice expressing PARK14 mutant (D331Y) PLA2G6, human (D331Y; GAC→TAC) mutation was introduced into the exon 7 of mouse PLA2G6 gene by knockin target vector-mediated homologous recombination. The resultant heterozygous PLA2G6^{WT/D331Y} knockin mice were bred and intercrossed to generate wild-type mice, heterozygous PLA2G6^{WT/D331Y} mice and homozygous PLA2G6^{D331Y/D331Y} knockin mice (FIG. 1A). RT-PCR assay using total RNA purified from SN and Sanger sequencing were performed to confirm (D331Y) PLA2G6 mutation (FIG. 1B).

PLA2G6 is localized in the cytosol and mitochondria. Cytosolic protein expression of PLA2G6 in the SN of PLA2G6^{WT/D331Y} or PLA2G6^{D331Y/D331Y} mice was similar to that of PLA2G6 in the SN of WT mice (FIG. 1C). Mitochondrial protein expression of PLA2G6 in the SN of PLA2G6^{WT/D331Y} or PLA2G6^{D331Y/D331Y} mice was also not significantly altered (FIG. 1C). Compared to the enzyme activity of PLA2G6 in the SN of wild-type mice, heterozygous or homozygous (D331Y) PLA2G6 mutation led to a significant decrease in PLA2G6 enzyme activity (FIG. 1D). These results indicated that (D331Y) PLA2G6 mutation causes the loss of function.

### Example 2

### PLA2G6^{D331Y/D331Y} KI Mice Display Early-Onset Degeneration of SNpc Dopaminergic Neurons

Homozygous (D331Y) PLA2G6 mutation causes early-onset autosomal-recessive PD. Immunohistochemical tyrosine hydroxylase (TH) staining was performed using 3- to 9-month-old heterozygous or homozygous (D331Y) PLA2G6 mice. The number of TH⁺-SNpc dopaminergic cells of PLA2G6^{D331Y/D331Y} knockin mice at the age of 3 months was not significantly different from that of age-matched PLA2G6^{WT/D331Y} or WT mice (FIG. 2A and 2C). Compared with WT mice, 6- or 9-month-old homozygous PLA2G6^{D331Y/D331Y} mice exhibited a significant reduction in the number of TH⁺-SNpc dopaminergic cells (FIGS. 2A and 2C). PLA2G6^{WT/D331Y} did not display cell death of TH+-SNpc dopaminergic neurons (FIGS. 2A and 2C). Six- or nine-month-old PLA2G6^{D331Y/D331Y} mice exhibited a significant reduction in the number of Niss1⁺-cells of SNpc (FIG. 2D and FIG. S1) Immunocytochemical staining of NeuN, a neuronal marker, demonstrated that neuronal loss was not found in the striatum (FIG. 2B and 2E), hippocampus and cerebral cortex (FIG. S2A and S2B) of PLA2G6^{D331Y/D331Y} mice.

PLA2G6^{D331Y/D331Y} knockin mice display the loss of nigrostriatal dopaminergic terminals. The radiotracer ¹⁸F-FP-DTBZ binds to vesicular monoamine transporter type 2. PET imaging with ¹⁸F-FP-DTBZ was conducted to monitor the integrity of nigrostriatal dopaminergic terminals in animal model of PD and PD patients. The microPET imaging analysis demonstrated that 3-month-old PLA2G6^{D331Y/D331Y} KI mice did not display a significant decrease in the striatal uptake of ¹⁸F-FP-DTBZ (FIG. 2F). Compared to 6- or 9-month-old WT mice, the striatal uptake of ¹⁸F-FP-DTBZ was significantly reduced in age-matched PLA2G6^{D331Y/D331Y} KI mice (FIG. 2F). Compared to 9-month-old WT mice, the density of striatal TH staining in age-matched PLA2G6^{D331Y/D331Y} mice was significantly decreased (FIG. 2G).

### Example 3

### PLA2G6^{D331Y/D331Y} KI Mice Exhibit Lewy Body-Like Pathology

Lewy body, which is mainly composed of α-synuclein (αSyn), phosphorylated α-synuclein and other components, is the neuropathological hallmark of Parkinson's disease Immunohistochemical staining using anti-phospho-α-synuclein^{Ser129} (p-αSyn) (FIG. 3A-3C) antiserum and anti-αSyn antibody (FIG. 3D-3F) demonstrated that Lewy bodies were found in the SN of PLA2G6^{D331Y/D331Y} mice at the age of 9 months Immunoblotting analysis demonstrated that PLA2G6^{D331Y/D331Y} KI mice at the age of 9 months displayed an upregulated protein expression of αSyn and p-αSyn (FIG. 3G) Immunofluorescence staining showed that the formation of αSyn aggregates was observed in the TH⁺-SN dopaminergic neuron of PLA2G6^{D331Y/D331Y} mice. Lewy body-like inclusions can be biochemically analyzed in sarkosyl-insoluble fraction. To confirm α-synuclein pathology, the accumulation of αSyn and p-αSyn was examined in the sarosyl-insoluble fraction obtained from the SN of (D331Y) PLA2G6 KI mice. Biochemical analysis showed that accumulation of αSyn and p-αSyn was observed in the sarosyl-insoluble fractions of homozygous (D331Y) PLA2G6 KI mice. PD patients with PLA2G6 mutations exhibit an increased protein level of hyperphosphorylated tau, which is the tau pathology. Western blot analysis indicated that protein expression of phospho-tau^{Ser202/Thr205} was upregulated in the substantia nigra of PLA2G6^{D331Y/D331Y} mice (FIG. 3H).

### Example 4

### PLA2G6^{D331Y/D331Y} Knockin Mice Exhibit Early-Onset and Progressive Parkinsonism Phenotypes

Six- to twelve-month-old PLA2G6^{D331Y/D331Y} mice exhibited an early-onset and progressive decrease in the locomotion activity, including the velocity (FIG. 4A) and distance (FIG. 4B). The cylinder test was conducted to analyze spontaneous movements of mice. Six- to twelve-month-old PLA2G6^{D331Y/D331Y} mice displayed a significant decrease in the number of rears (FIG. 4C). Motor coordination of animal was evaluated by performing the rotarod test. Six- to twelve-month old PLA2G6^{D331Y/D331Y} knockin mice displayed an impaired motor coordination, which was indicated by a marked reduction in retention time on the rotarod apparatus (FIG. 4D). The pole test was conducted to examine the motor performance of animal. Six- to twelve-month old PLA2G6^{D331Y/D331Y} KI mice displayed an increase in time required to perform the pole test, which indicated impaired motor performance and bradykinesia phenotype (FIG. 4E).

L-DOPA was effective in treating PD patients carrying PLA2G6 mutations. Similar to PARK14 patients, treatment of methyl L-DOPA rescued hypoactivity displayed by 9-month-old PLA2G6^{D331Y/D331Y} knockin mice (FIGS. 4F and 4G).

### Example 5

### Homozygous (D331Y) PLA2G6 KI Mice Exhibit Mitochondrial Degeneration and Mitochondrial Dysfunction

The ultrastructure of mitochondria in the neuromelanin organelle-containing SNpc dopaminergic neuron was examined by performing transmission electron microscopy. Intact morphology and well-defined cristae of mitochondria were observed in the neuromelanin-positive putative SNpc dopaminergic cells of WT mice or heterozygous PLA2G6^{WT/D331Y} mice (n=20 neurons). In contrast, disrupted cristae of mitochondria were found in the neuromelanin organelle-containing putative SN dopaminergic neurons of PLA2G6^{D331Y/D331Y} knockin mice (n=20 neurons) (FIG. 5A-C). Moreover, the mitochondrial size was decreased in the SN of homozygous (D331Y) PLA2G6 mice compared to WT or heterozygous (D331Y) PLA2G6 mice (FIG. 5D).

Abnormal ultrastructure of mitochondria could lead to mitochondrial dysfunction. An impaired activity of mitochondrial complex I activity is found in PD patients. The activities of mitochondrial complex I-IV were analyzed in the SN of homozygous (D331Y) PLA2G6 KI mice. Compared with WT mice, the activity of mitochondrial complex I or III was reduced in the SN of PLA2G6^{D331Y/D331Y} mice (FIGS. 5E and 5G). The activity of mitochondrial complex II or IV was not significantly altered in the SN of PLA2G6^{D331Y/D331Y} mice (FIGS. 5F and 5H). A reduced level of intracellular ATP production was also found in the SN of PLA2G6^{D331Y/D331Y} mice (FIG. 5I).

Mitochondrial dysfunction causes the overproduction of reactive oxygen species (ROS) and lipid peroxidation. Cardiolipin, a unique mitochondrial phospholipid, participates in maintaining mitochondrial function and cytochrome c release. ROS generated from mitochondria results in the oxidation of cardiolipin and subsequent activation of apoptosis through the release of cytochrome c. The status of cardiolipin oxidation can be determined by measuring the lipid peroxidation of mitochondria and the release of cytochrome c simultaneously.

Overproduction of mitochondrial ROS was observed in the SN of PLA2G6^{D331Y/D331Y} mice (FIG. 5J). Lipid peroxidation of mitochondria was evaluated by performing thiobarbituric acid reactive substances assay. An increased level of lipid peroxidation was found in the SN of PLA2G6^{D331Y/D331Y} mice (FIG. 5K). Cytosolic level of cytochrome c was also markedly upregulated in the SN of PLA2G6^{D331Y/D331Y} mice (FIG. 5L).

### Example 6

### Homozygous (D331Y) PLA2G6 Mutation Causes the Activation of Mitochondrial Apoptotic Pathway, the Induction of Endoplasmic Reticulum Stress and Mitophagy Dysfunction

To test the possibility that the activation of mitochondrial apoptotic pathway causes cell death of SN dopaminergic neurons observed in PLA2G6^{D331Y/D331Y} mice, apoptotic proteins were evaluated in the SN of WT or KI mice. The level of cytosolic cytochrome c, active caspase-9 or active caspase-3 was significantly increased in the SN of PLA2G6^{D331Y/D331Y} mice at the age of 9 months (FIG. 6A).

Endoplasmic reticulum (ER) stress is implicated in the pathogenesis of PD. Mitochondrial dysfunction results in overproduction of ROS, which then gives rise to ER stress. To determine whether homozygous (D331Y) PLA2G6 mutation triggers ER stress, protein levels of ER stress-related proteins were examined in the SN of WT or KI mice. Protein levels of 78-kDa glucose regulated protein (Grp78), inositol requiring enzyme 1α (IRE1α), protein kinase RNA-like ER kinase (PERK) and C/EBP homologous protein (CHOP), which participate in the activation of ER stress, were increased in the SN of PLA2G6^{D331Y/D331Y} mice.

Impaired mitophagy plays an important role in the pathogenesis of PD Immunoblotting analysis demonstrated that protein levels of parkin and BNIP3, which are autophagy/mitophagy-related proteins, were decreased in the SN of PLA2G6^{D331Y/D331Y} knockin mice (FIG. 6C).

### Example 7

### (D331Y) PLA2G6 Alters mRNA Levels of Genes, Which Possess Neurotoxic or Neuroprotective Effect, in the SN of PLA2G6^{D331Y/D331Y} Knockin Mice

In addition to participating in maintaining mitochondrial function, PLA2G6 also participates in gene regulation. Therefore, mutant (D331Y) PLA2G6-induced transcriptional dysregulation is also likely to be involved in (D331Y) PLA2G6-induced neuronal death of SNpc dopaminergic cells. To test this possibility, transcriptomic analysis was conducted to evaluate differential mRNA expressions in the SN of PLA2G6^{DD331Y/D331Y} mice at the age of 9 months. The heat map produced by hierarchical gene clustering displayed a different pattern between WT mice and PLA2G6^{D331Y/D331Y} mice (FIG. 7A).

Microarray analysis indicated that the mRNA expression of microtubule affinity-regulating kinase 4 (Mark4), which causes neurotoxic effects, and XIAP associated factor 1 (Xaf1), which participates in the induction of apoptosis, was increased in the SN of PLA2G6^{D331Y/D331Y} KI mouse. The mRNA expression of bone morphogenetic protein 6 (Bmp6), cyclin D2 (Ccnd2), beta-catenin 1 (Ctnnb1), heat shock protein 1B (Hspa1b), mitogen-activated protein kinase 1 (Mapk1), kinase D-interacting substrate 220 (Kidins220), prosaposin (Psap) and syndecan 2 (Sdc2), which exert a neuroprotective effect, was downregulated in the SN of PLA2G6^{D331Y/D331Y} mice (FIG. 7A).

Consistent with results of microarray analysis, quantitative RT-PCR analysis demonstrated that mRNA level of Mark4 or Xaf1 was significantly increased in the SN of PLA2G6^{D331Y/D331Y} mice (FIG. 7B). The mRNA expression of Bmp6, Ccnd2, Ctnnb1, Hspa1b, Mapk1, Kidins220, Psap and Sdc2 was downregulated in the SN of PLA2G6^{DD331Y/D331Y} mice (FIG. 7B).

To provide the evidence that prior to a significant neurodegeneration of SNpc dopaminergic cells, differential mRNA expression of the above 10 genes is observed at the earlier phase of disease, WT or PLA2G6^{D331Y/D331Y} mice at the age of 5 months, which did not exhibit a significant degeneration of SNpc dopaminergic cells, were used to conduct quantitative RT-PCR analysis. An upregulated mRNA level of Mark4 or Xaf1and a downregulated mRNA level of Bmp6, Ccnd2, Ctnnb1, Hspa1b, Mapk1, Kidins220, Psap or Sdc2 were observed in the SN of PLA2G6^{D331Y/D331Y} mice at the age of 5 months (FIG. 7C).

Consistent with the results of transcriptomic analysis and quantitative RT-PCR assays, western blot study demonstrated that protein expression of Mark4 or Xaf1 was upregulated in the SN of PLA2G6^{D331Y/D331Y} mice at the age of 9 months (FIGS. 7D and 7E). Protein expression of Bmp6, Ccnd2, Ctnnb1, Hspa1b, Mapk1, Kidins220, Psap or Sdc2 was also downregulated in the SN of PLA2G6^{D331Y/D331Y} mice (FIGS. 7D and 7E).

### Example 8

### Detection of Nucleotide Mutation at Position 991 from G to T in PLA2G6 Gene

Genomic DNA was extracted from a blood sample of patients suspected of suffering from early-onset PD (PARK14).

One of the following pairs of forward (F) primer and reverse (R) primer was used for performing PCR amplification of a coding region of PLA2G6 gene:

```
(SEQ ID NO. 1)
17) F primer: gacagggccaccagtgattg;
(SEQ ID NO. 2)
        R primer: agttcgagatgagacacgggc;
(SEQ ID NO. 3)
18) F primer: caggatctggggacaacgc;
(SEQ ID NO. 4)
        R primer: gccaataagacctccaatcc;
(SEQ ID NO. 5)
19) F primer: gggaccttctgattccagc;
(SEQ ID NO. 6)
        R primer: gcccacacaagcaggtacac;
(SEQ ID NO. 7)
20) F primer: aaagtccgagtttccgagtg;
(SEQ ID NO. 8)
        R primer: aggcctgagagtgacacctg;
(SEQ ID NO. 9)
21) F primer: cccggcctctttacgttc;
(SEQ ID NO. 10)
        R primer: ctcaggcacgggacagg;
(SEQ ID NO. 11)
22) F primer: cttcatcccacgccacg;
(SEQ ID NO. 12)
        R primer: gaacctgcttcctgaggg;
(SEQ ID NO. 13)
23) F primer: cagtgcccacgtgtccc;
(SEQ ID NO. 14)
        R primer: gacagccctcctgcattc;
(SEQ ID NO. 15)
24) F primer: ctttgttcttcacttccccg;
(SEQ ID NO. 16)
        R primer: ctcggtccctgtatccacc;
(SEQ ID NO. 17)
25) F primer: agctgcttgggatgtaccagc;
(SEQ ID NO. 18)
        R primer: cggcttcctttagtgacttccg;
(SEQ ID NO. 19)
26) F primer: ctagggacctctggggtagc;
(SEQ ID NO. 20)
        R primer: gtgaggggcaggaaagc;
(SEQ ID NO. 21)
27) F primer: aaagtactgggctgtggcag;
(SEQ ID NO. 22)
        R primer: gcaaagccctgaagacaaac;
(SEQ ID NO. 23)
28) F primer: aatttgggtttgcttaggcctc;
(SEQ ID NO. 24)
        R primer: gttccctctgctcccctcaag;
(SEQ ID NO. 25)
29) F primer: aattgtggggaaagggaaag;
(SEQ ID NO. 26)
        R primer: accaccccacagcctctc;
(SEQ ID NO. 27)
30) F primer: catgggttttatgccagtcc;
(SEQ ID NO. 28)
        R primer: gtccctagcatggtttgctg;
(SEQ ID NO. 29)
31) F primer: ccccagagcccagtcttg;
(SEQ ID NO. 30)
        R primer: gtctcctccaacaccaaagg; or
(SEQ ID NO. 31)
32) F primer: gctccgagagtgcaggg;
(SEQ ID NO. 32)
        R primer: gcaggggctgaatggac.
```

The following materials and reagents were used for performing said PCR amplification:

Genomic DNA extracted from a blood sample of patients suspected of suffering from early-onset

| PD (PARK14) (100 μg/μl) | | 1 μl |
|---|---|---|
| F primer (10 μM) | | 1 μl |
| R primer (10 μM) | | 1 μl |
| 10X PCR buffer | | 2.5 μ1 |
| Qiagen HotStarTaq DNA polymerase | | 0.5 μl |
| Deionized water | added to | 25 μl |

The steps and conditions for performing said PCR amplification were shown in FIG. 8(A).

A PLA2G6 gene mutation pattern was then determined by sequence analysis.

If the PLA2G6 gene mutation pattern comprises a nucleotide mutation of G991T, then the patients can be recognized as ones suffering from early-onset PD (PARK14) (FIG. 8(B)).

1. A homozygous PLA2G6^{D331Y/D331Y} knockin mouse that simultaneously displays early-onset degeneration of dopaminergic neurons in substantia nigra pars compacta (SNpc), synucleinopathy and tau pathology.

2. The homozygous PLA2G6^{D331Y/D331Y} knockin mouse according to claim 1, wherein the degeneration of dopaminergic neurons in substantia nigra pars compacta (SNpc), synucleinopathy and tau pathology occur within about 6 to about 9 months.

3. The homozygous PLA2G6^{D331Y/D331Y} knockin mouse according to claim 1, wherein in the dopaminergic neurons, the structure of mitochondria cristae is disrupted, and the activity of mitochondrial complex I is reduced.

4. The homozygous PLA2G6^{D331Y/D331Y} knockin mouse according to claim 1, wherein in the dopaminergic neurons, the enzyme activity of PLA2G6 is reduced.

5. The homozygous PLA2G6^{D331Y/D331Y} knockin mouse according to claim 1, wherein the dopaminergic neurons displays mitochondrial mitophagy dysfunction.

6. A platform for screening of active agents for treating early-onset Parkinson's disease, comprising the homozygous PLA2G6^{D331Y/D331Y} knockin mouse according to claim 1.

7. A method of using the platform according to claim 6 for screening of active agents for treating early-onset Parkinson's disease, comprising the steps of (1) administering a candidate agent to the homozygous PLA2G6^{D331Y/D331Y} knockin mouse, and (2) evaluating effects of the candidate agent in improving symptoms of motor dysfunction or motor deficit in said homozygous PLA2G6^{D331Y/D331Y} knockin mouse.

8. The method according to claim 7, wherein the effects in improving symptoms of motor dysfunction or motor deficit include: increased level of PLA2G6, reduced degeneration or death of dopaminergic neurons, reduced synucleinopathy and tau pathology, reduced accumulation of Lewy bodies, increased level of mitochondrial complex I, increased ATP synthesis, reduced level of ROS, increased expression level of growth- and protection-related proteins of dopaminergic neurons, reduced expression of proteins relating to apoptosis of dopaminergic neurons, and increased expression of Catenin beta-1.

9. The method according to claim 8, wherein the growth- and protection-related proteins of dopaminergic neurons include one or more of Bmp6, Ccnd2, Ctnnb1, Hspa1b, Kidins220, Mapk1, Psap and Sdc2.

10. The method according to claim 8, wherein the proteins relating to apoptosis of dopaminergic neurons include one or both of Mark4 and Xaf1.

11. A kit of detecting the presence of a nucleotide mutation of G991T in the coding region of PLA2G6 gene for diagnosing early-onset Parkinson's disease in a subject, comprising:
(1) a pair of forward (F) primer and reverse (R) primer selected from the followings:
```
(SEQ ID NO. 1)
  1) F primer: gacagggccaccagtgattg;
(SEQ ID NO. 2)
        R primer: agttcgagatgagacacgggc;
(SEQ ID NO. 3)
  2) F primer: caggatctggggacaacgc;
(SEQ ID NO. 4)
        R primer: gccaataagacctccaatcc;
(SEQ ID NO. 5)
  3) F primer: gggaccttctgattccagc;
(SEQ ID NO. 6)
        R primer: gcccacacaagcaggtacac;
(SEQ ID NO. 7)
  4) F primer: aaagtccgagtttccgagtg;
(SEQ ID NO. 8)
        R primer: aggcctgagagtgacacctg;
(SEQ ID NO. 9)
  5) F primer: cccggcctctttacgttc;
(SEQ ID NO. 10)
        R primer: ctcaggcacgggacagg;
(SEQ ID NO. 11)
  6) F primer: cttcatcccacgccacg;
(SEQ ID NO. 12)
        R primer: gaacctgcttcctgaggg;
(SEQ ID NO. 13)
  7) F primer: cagtgcccacgtgtccc;
(SEQ ID NO. 14)
        R primer: gacagccctcctgcattc;
(SEQ ID NO. 15)
  8) F primer: ctttgttcttcacttccccg;
(SEQ ID NO. 16)
        R primer: ctcggtccctgtatccacc;
(SEQ ID NO. 17)
  9) F primer: agctgcttgggatgtaccagc;
(SEQ ID NO. 18)
        R primer: cggcttcctttagtgacttccg;
(SEQ ID NO. 19)
10) F primer: ctagggacctctggggtagc;
(SEQ ID NO. 20)
        R primer: gtgaggggcaggaaagc;
(SEQ ID NO. 21)
11) F primer: aaagtactgggctgtggcag;
(SEQ ID NO. 22)
        R primer: gcaaagccctgaagacaaac;
(SEQ ID NO. 23)
12) F primer: aatttgggtttgcttaggcctc;
(SEQ ID NO. 24)
        R primer: gttccctctgctcccctcaag;
(SEQ ID NO. 25)
13) F primer: aattgtggggaaagggaaag;
(SEQ ID NO. 26)
        R primer: accaccccacagcctctc;
(SEQ ID NO. 27)
14) F primer: catgggttttatgccagtcc;
(SEQ ID NO. 28)
        R primer: gtccctagcatggtttgctg;
(SEQ ID NO. 29)
15) F primer: ccccagagcccagtcttg;
(SEQ ID NO. 30)
        R primer: gtctcctccaacaccaaagg; or
(SEQ ID NO. 31)
16) F primer: gctccgagagtgcaggg;
(SEQ ID NO. 32)
        R primer: gcaggggctgaatggac;
```
(2) a buffer;
(3) PCR polymerase; and
(4) an instruction for use,
wherein the instruction use describes the steps of:
(a) extracting genomic DNA from a blood sample of patients suspected of suffering from early-onset Parkinson's disease;
(b) performing PCR amplification of a coding region of PLA2G6 gene by using a pair of F primer and R primer as shown in the above item (1); and
(c) sequencing the DNA fragments obtained from the PCR amplification, and aligning for comparison the sequenced DNA fragments with the correspondent sequenced DNA fragments obtained from a healthy subject,
wherein, if there is nucleotide G991T mutation in the coding region of PLA2G6 gene, then the patients can be recognized as ones suffering from early-onset Parkinson's disease.


SEQUENCE LISTING (32 sequences)

SEQ ID NO: 1 - DNA, 20 bp, Artificial Sequence
  Primer
gacagggccaccagtgattg

SEQ ID NO: 2 - DNA, 21 bp, Artificial Sequence
  Primer
agttcgagatgagacacgggc

SEQ ID NO: 3 - DNA, 19 bp, Artificial Sequence
  Primer
caggatctggggacaacgc

SEQ ID NO: 4 - DNA, 20 bp, Artificial Sequence
  Primer
gccaataagacctccaatcc

SEQ ID NO: 5 - DNA, 19 bp, Artificial Sequence
  Primer
gggaccttctgattccagc

SEQ ID NO: 6 - DNA, 20 bp, Artificial Sequence
  Primer
gcccacacaagcaggtacac

SEQ ID NO: 7 - DNA, 20 bp, Artificial Sequence
  Primer
aaagtccgagtttccgagtg

SEQ ID NO: 8 - DNA, 20 bp, Artificial Sequence
  Primer
aggcctgagagtgacacctg

SEQ ID NO: 9 - DNA, 18 bp, Artificial Sequence
  Primer
cccggcctctttacgttc

SEQ ID NO: 10 - DNA, 17 bp, Artificial Sequence
  Primer
ctcaggcacgggacagg

SEQ ID NO: 11 - DNA, 17 bp, Artificial Sequence
  Primer
cttcatcccacgccacg

SEQ ID NO: 12 - DNA, 18 bp, Artificial Sequence
  Primer
gaacctgcttcctgaggg

SEQ ID NO: 13 - DNA, 17 bp, Artificial Sequence
  Primer
cagtgcccacgtgtccc

SEQ ID NO: 14 - DNA, 18 bp, Artificial Sequence
  Primer
gacagccctcctgcattc

SEQ ID NO: 15 - DNA, 20 bp, Artificial Sequence
  Primer
ctttgttcttcacttccccg

SEQ ID NO: 16 - DNA, 19 bp, Artificial Sequence
  Primer
ctcggtccctgtatccacc

SEQ ID NO: 17 - DNA, 21 bp, Artificial Sequence
  Primer
agctgcttgggatgtaccagc

SEQ ID NO: 18 - DNA, 22 bp, Artificial Sequence
  Primer
cggcttcctttagtgacttccg

SEQ ID NO: 19 - DNA, 20 bp, Artificial Sequence
  Primer
ctagggacctctggggtagc

SEQ ID NO: 20 - DNA, 17 bp, Artificial Sequence
  Primer
gtgaggggcaggaaagc

SEQ ID NO: 21 - DNA, 20 bp, Artificial Sequence
  Primer
aaagtactgggctgtggcag

SEQ ID NO: 22 - DNA, 20 bp, Artificial Sequence
  Primer
gcaaagccctgaagacaaac

SEQ ID NO: 23 - DNA, 22 bp, Artificial Sequence
  Primer
aatttgggtttgcttaggcctc

SEQ ID NO: 24 - DNA, 21 bp, Artificial Sequence
  Primer
gttccctctgctcccctcaag

SEQ ID NO: 25 - DNA, 20 bp, Artificial Sequence
  Primer
aattgtggggaaagggaaag

SEQ ID NO: 26 - DNA, 18 bp, Artificial Sequence
  Primer
accaccccacagcctctc

SEQ ID NO: 27 - DNA, 20 bp, Artificial Sequence
  Primer
catgggttttatgccagtcc

SEQ ID NO: 28 - DNA, 20 bp, Artificial Sequence
  Primer
gtccctagcatggtttgctg

SEQ ID NO: 29 - DNA, 18 bp, Artificial Sequence
  Primer
ccccagagcccagtcttg

SEQ ID NO: 30 - DNA, 20 bp, Artificial Sequence
  Primer
gtctcctccaacaccaaagg

SEQ ID NO: 31 - DNA, 17 bp, Artificial Sequence
  Primer
gctccgagagtgcaggg

SEQ ID NO: 32 - DNA, 17 bp, Artificial Sequence
  Primer
gcaggggctgaatggac